(19) United States
(12) Patent Application Publication
(10) Pub. No.: US 2015/0224085 A1
(43) Pub. Date: Aug. 13, 2015

(54) METHOD OF ADMINISTERING A 5,5-FUSED HETEROARYLENE HEPATITIS C VIRUS INHIBITOR FOR TREATING OF PREVENTING HEPATITIS C VIRUS INFECTION

(71) Applicant: Idenix Pharmaceuticals, Inc., Cambridge, MA (US)

(72) Inventors: Rahela Gasparac, Watertown, MA (US); Benjamin Mayes, Boston, MA (US); Adel Moussa, Burlington, MA (US); Keith Piet-Ropaolo, Boston, MA (US); John Sullivan-Bolyai, Westport Point, MA (US); Xiao-Jian Zhou, Arlington, MA (US)

(73) Assignee: Indenix Pharmaceuticals, Inc, Cambridge, MA (US)

(21) Appl. No.: 14/424,968

(22) PCT Filed: Aug. 29, 2013
(86) PCT No.: PCT/US2013/057270

§ 371 Date: Feb. 27, 2015

Related U.S. Application Data
(60) Provisional application No. 61/696,022, filed on Aug. 31, 2012.
(60) Provisional application No. 61/721,374, filed on Nov. 1, 2012.

(51) Int. Cl. A61K 31/4184; A61K 45/06

(52) U.S. Cl. CPC A61K 31/4184; A61K 45/06

(57) ABSTRACT

Provided herein are methods of administering a 5,5-fused heteroarylene hepatitis C virus inhibitor compound or an isotopic variant thereof, or a pharmaceutically acceptable salt or solvate thereof; for treating or preventing hepatitis C virus infection in a subject.

### CROSS REFERENCE TO RELATED APPLICATIONS

This application claims priority to U.S. Provisional Application No. 61/696,022, filed Aug. 31, 2012; and 61/721,374, filed Nov. 1, 2012; the disclosure of each of which is incorporated herein by reference in its entirety.

### FIELD

Provided herein are methods of administering a 5,5-fused heteroarylene hepatitis C virus inhibitor compound or an isotopic variant thereof, or a pharmaceutically acceptable salt or solvate thereof; for treating or preventing hepatitis C virus infection in a subject.

### BACKGROUND

Hepatitis C virus (HCV) is known to cause at least 80% of posttransfusion hepatitis and a substantial proportion of sporadic acute hepatitis (Kuo et al., Science 1989, 244, 362-364; Thomas, Curr. Top. Microbiol. Immunol. 2000, 25-41). Preliminary evidence also implicates HCV in many cases of “idiopathic” chronic hepatitis, “cryptogenic” cirrhosis, and probably hepatocellular carcinoma unrelated to other hepatitis viruses, such as hepatitis B virus (Di Besceglie et al., Scientific American, 1999, October, 80-85; Boyer et al., J. Hepatol. 2000, 32, 98-112).

HCV is an enveloped virus containing a positive-sense single-stranded RNA genome of approximately 9.4 kb (Kato et al., Proc. Natl. Acad. Sci. USA 1990, 87, 9524-9528; Kato, Acta Medica Okayama, 2001, 55, 133-159). The viral genome consists of a 5′ untranslated region (UTR), a long open reading frame encoding a polyprotein precursor of approximately 3011 amino acids, and a short 3′ UTR. The 5′ UTR is the most highly conserved part of the HCV genome and is important for the initiation and control of polyprotein translation. Translation of the HCV genome is initiated by a cap-independent mechanism known as an internal ribosome entry. This mechanism involves the binding of ribosomes to an RNA sequence known as the internal ribosome entry site (IRES). An RNA pseudoknot structure has recently been determined to be an essential structural element of the HCV IRES. Viral structural proteins include a nucleocapsid core protein (C) and two envelope glycoproteins, E1 and E2. HCV also encodes two proteinases, a zinc-dependent metalloproteinase encoded by the NS2-NS3 region and a serine proteinase encoded in the NS3 region. These proteinases are required for cleavage of specific regions of the precursor polyprotein into mature peptides. The carboxyl half of nonstructural protein 5, NS5B, contains the RNA-dependent RNA polymerase. The function of the remaining nonstructural proteins, NS4A and NS4B, and that of NS5A (the amino-terminal half of nonstructural protein 5) remain unknown.

Presently, the most effective HCV therapy employs a combination of alpha-interferon and ribavirin, leading to sustained efficacy in about 40% of patients (Poynard et al., Lancet 1998, 352, 1426-1432). Recent clinical results demonstrate that pegylated alpha-interferon is superior to unmodified alpha-interferon as monotherapy. However, even with experimental therapeutic regimens involving combinations of pegylated alpha-interferon and ribavirin, a substantial fraction of patients do not have a sustained reduction in viral load (Manns et al., Lancet 2001, 358, 958-965; Fried et al., N. Engl. J. Med. 2002, 347, 975-982; Hadziyannis et al., Ann. Intern. Med. 2004, 140, 346-355). Thus, there is a clear and unmet need to develop effective therapeutics for treatment of HCV infection.

### SUMMARY OF THE DISCLOSURE

Provided herein is a method for treating or preventing a hepatitis C virus infection in a subject, comprising administering to the subject a therapeutically effective amount of [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester or an isotopic variant thereof; or a pharmaceutically acceptable salt or solvate thereof; wherein the therapeutically effective amount is at least 1 mg per day.

Also provided herein is a method for treating or preventing a hepatitis C virus infection in a subject, comprising administering to the subject a therapeutically effective amount of [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester or an isotopic variant thereof; or a pharmaceutically acceptable salt or solvate thereof; wherein the therapeutically effective amount is from about 0.02 to about 20 mg/kg/day.

Additionally, provided herein is a method for treating or preventing a hepatitis C virus infection in a subject, comprising administering to the subject [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester or an isotopic variant thereof; or a pharmaceutically acceptable salt or solvate thereof; in an amount that is sufficient to provide a plasma concentration of the compound at steady state in the range from about 1 nM to about 1 μM.

Furthermore, provided herein is a method for treating or preventing a hepatitis C virus infection in a subject, comprising administering to the subject [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester or an isotopic variant thereof; or a pharmaceutically acceptable salt or solvate thereof; in an amount that is sufficient to provide a peak plasma concentration of the compound ranging from about 5 nM to about 1 μM.

Provided herein is a method for treating or preventing a hepatitis C virus infection in a subject, comprising administering to the subject [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester or an isotopic variant thereof; or a pharmaceutically acceptable salt or solvate thereof; in an amount that is sufficient to provide a trough plasma concentration of the compound ranging from about 1 nM to about 500 nM.

Provided herein is a method for treating or preventing a hepatitis C virus infection in a subject, comprising administering to the subject [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester or an isotopic variant thereof; or a pharmaceutically acceptable salt or solvate thereof; in an amount that is sufficient to provide an area under the curve (AUC) of the compound in the range from about 100 to about 10,000 ng·hr/mL.

Provided herein is a method for treating, preventing, or ameliorating one or more symptoms of a liver disease or disorder associated with an HCV infection in a subject, comprising administering to the subject a therapeutically effective amount of [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester or an isotopic variant thereof; or a pharmaceutically acceptable salt or solvate thereof; wherein the therapeutically effective amount is at least 1 mg per day.

Provided herein is a method for treating, preventing, or ameliorating one or more symptoms of a liver disease or disorder associated with an HCV infection in a subject, comprising administering to the subject a therapeutically effective amount of [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester or an isotopic variant thereof; or a pharmaceutically acceptable salt or solvate thereof; wherein the therapeutically effective amount is from about 0.02 to about 20 mg/kg/day.

Provided herein is a method for treating, preventing, or ameliorating one or more symptoms of a liver disease or disorder associated with an HCV infection in a subject, comprising administering to the subject [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester or an isotopic variant thereof; or a pharmaceutically acceptable salt or solvate thereof; in an amount that is sufficient to provide a plasma concentration of the compound at steady state in the range from about 1 nM to about 1 μM.

Provided herein is a method for treating, preventing, or ameliorating one or more symptoms of a liver disease or disorder associated with an HCV infection in a subject, comprising administering to the subject [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester or an isotopic variant thereof; or a pharmaceutically acceptable salt or solvate thereof; in an amount that is sufficient to provide a peak plasma concentration of the compound ranging from about 5 nM to about 1 μM.

Provided herein is a method for treating, preventing, or ameliorating one or more symptoms of a liver disease or disorder associated with an HCV infection in a subject, comprising administering to the subject [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester or an isotopic variant thereof; or a pharmaceutically acceptable salt or solvate thereof; in an amount that is sufficient to provide a trough plasma concentration of the compound ranging from about 1 nM to about 500 nM.

Provided herein is a method for treating, preventing, or ameliorating one or more symptoms of a liver disease or disorder associated with an HCV infection in a subject, comprising administering to the subject [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester or an isotopic variant thereof; or a pharmaceutically acceptable salt or solvate thereof; in an amount that is sufficient to provide an area under the curve (AUC) of the compound in the range from about 100 to about 10,000 ng·hr/mL.

Provided herein is a method for inhibiting replication of a virus in a subject, comprising administering to the subject a therapeutically effective amount of [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester or an isotopic variant thereof; or a pharmaceutically acceptable salt or solvate thereof; wherein the therapeutically effective amount is at least 1 mg per day.

Provided herein is a method for inhibiting replication of a virus in a subject, comprising administering to the subject a therapeutically effective amount of [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester or an isotopic variant thereof; or a pharmaceutically acceptable salt or solvate thereof; wherein the therapeutically effective amount is from about 0.02 to about 20 mg/kg/day.

Provided herein is a method for inhibiting replication of a virus in a subject, comprising administering to the subject [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester or an isotopic variant thereof; or a pharmaceutically acceptable salt or solvate thereof; in an amount that is sufficient to provide a plasma concentration of the compound at steady state in the range from about 1 nM to about 1 μM.

Provided herein is a method for inhibiting replication of a virus in a subject, comprising administering to the subject [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester or an isotopic variant thereof; or a pharmaceutically acceptable salt or solvate thereof; in an amount that is sufficient to provide a peak plasma concentration of the compound ranging from about 5 nM to about 1 μM.

Provided herein is a method for inhibiting replication of a virus in a subject, comprising administering to the subject [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester or an isotopic variant thereof; or a pharmaceutically acceptable salt or solvate thereof; in an amount that is sufficient to provide a trough plasma concentration of the compound ranging from about 1 nM to about 500 nM.

Provided herein is a method for inhibiting replication of a virus in a subject, comprising administering to the subject [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester or an isotopic variant thereof; or a pharmaceutically acceptable salt or solvate thereof; in an amount that is sufficient to provide an area under the curve (AUC) of the compound in the range from about 100 to about 10,000 ng·hr/mL.

### BRIEF DESCRIPTION OF THE DRAWINGS

FIG. 1 depicts the HCV RNA levels in the subjects having HCV Genotype 1a infection, treated with [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester.

FIG. 2 depicts the HCV RNA levels in the subjects having HCV Genotype 1b infection, treated with [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester.

FIG. 3 depicts the HCV RNA levels in the subjects having HCV Genotype 2 infection, treated with [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester.

FIG. 4 depicts the HCV RNA levels in the subjects having HCV Genotype 3 infection, treated with [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester.

FIG. 5 depict the relationship between the maximum viral response and plasma HCV RNA levels (C24h) after single doses of [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester in Genotypes 1a, 1b, 2, and 4 HCV-infected subjects.

FIG. 6 depicts the plasma concentrations of [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester over time at a dose of 5, 10, 25, 50, or 100 mg per day in healthy subjects.

FIG. 7 depicts the plasma concentrations of [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester over time at a dose of 1, 5, 10, 25, 50, or 100 mg per day in HCV-infected subjects.

FIG. 8 depicts the plasma concentrations of [(S)-1-((S)-2-({6-[6-(4-({(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester over time at a dose of 100 mg per day in HCV-infected subjects (HCV) and healthy subjects (HV).

FIG. 9 depict mean (+SE) changes from baseline HCV RNA in the subjects having HCV Genotype 1, 2, 3, or 4 infection, treated with [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester (“the Compound”): placebo: ———; 25 mg QD of the Compound: — — —; 50 mg QD of the Compound —-—; 50 mg BID of the Compound: —— —; 100 mg QD of the Compound: — —•.

FIG. 10 depict mean plasma concentrations of [(S)-1-((S)-2-({6-[6-(4-({(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester over time after QD and BID dosing in genotype 1 to 4 HCV-infected subjects on Days 1 and 3, respectively.

FIG. 11 depict changes of each individual subject from baseline HCV RNA in the subjects having HCV Genotype 2 infection, treated with [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester (“the Compound”): A—placebo; B—Treatment with 50 mg of the Compound BID; and C—Treatment with 100 mg of the Compound QD.

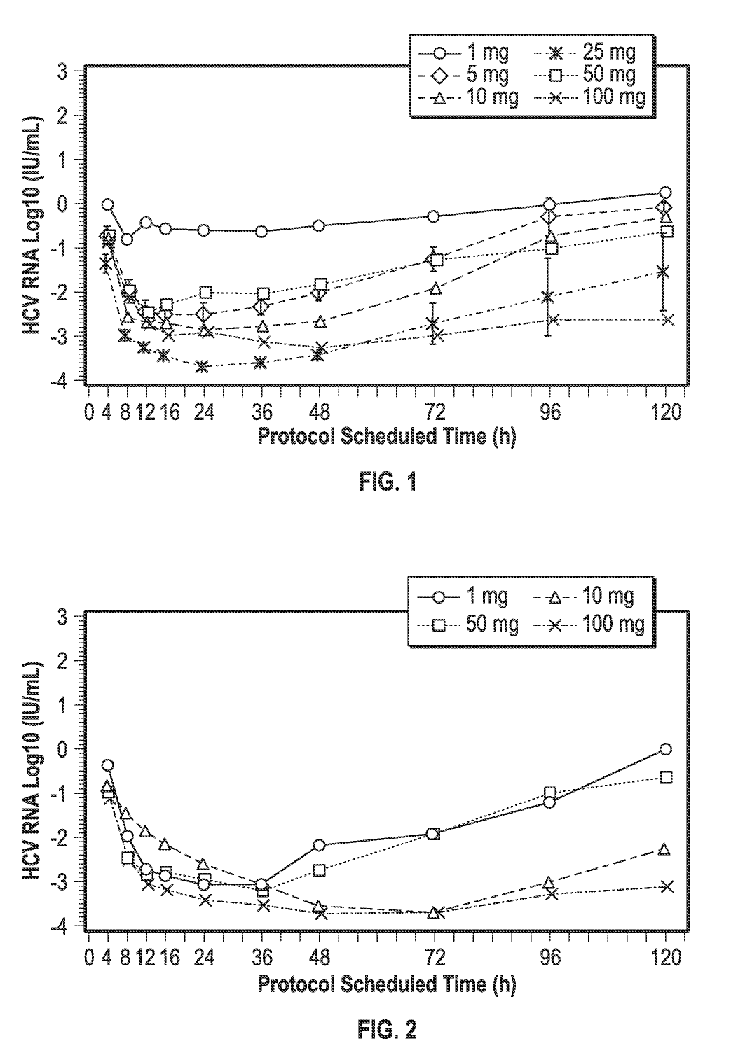

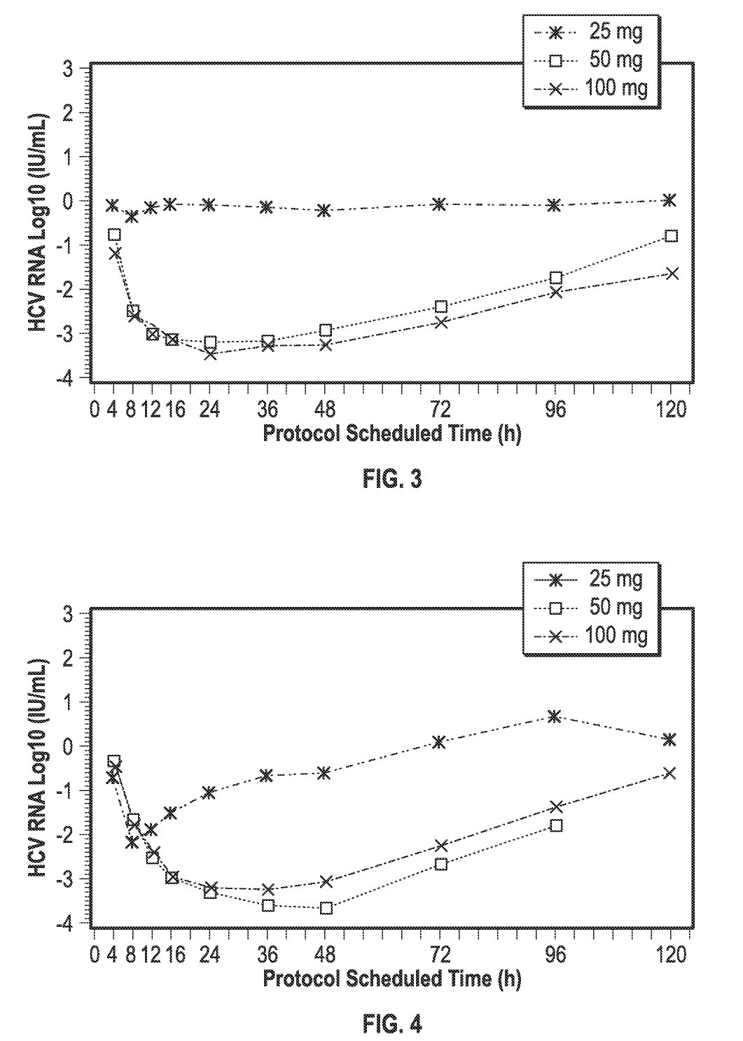

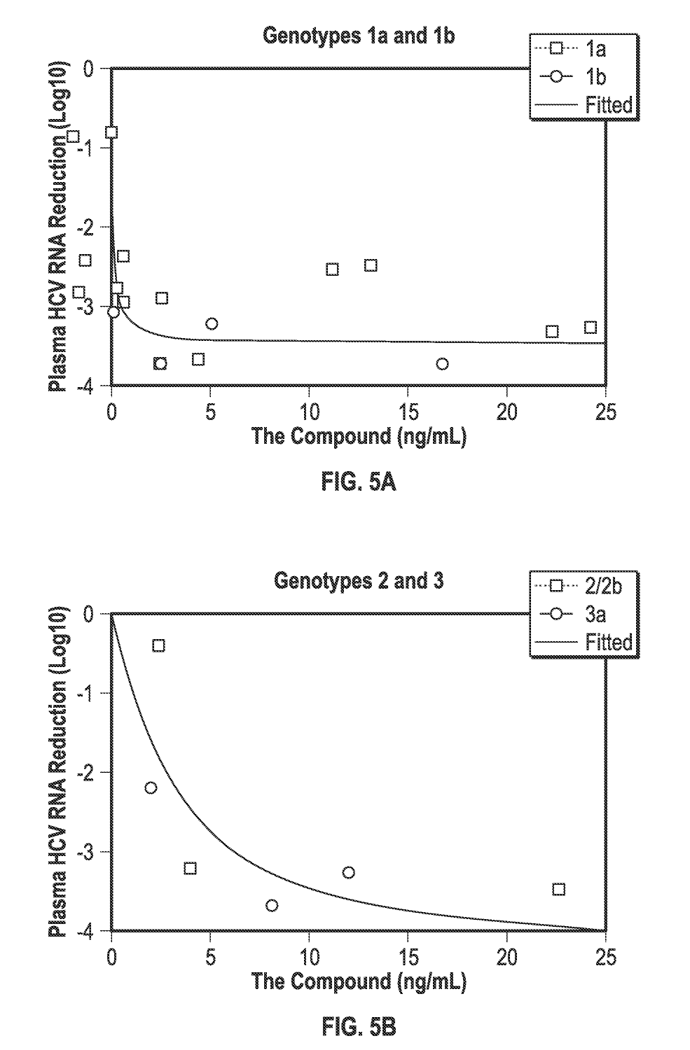

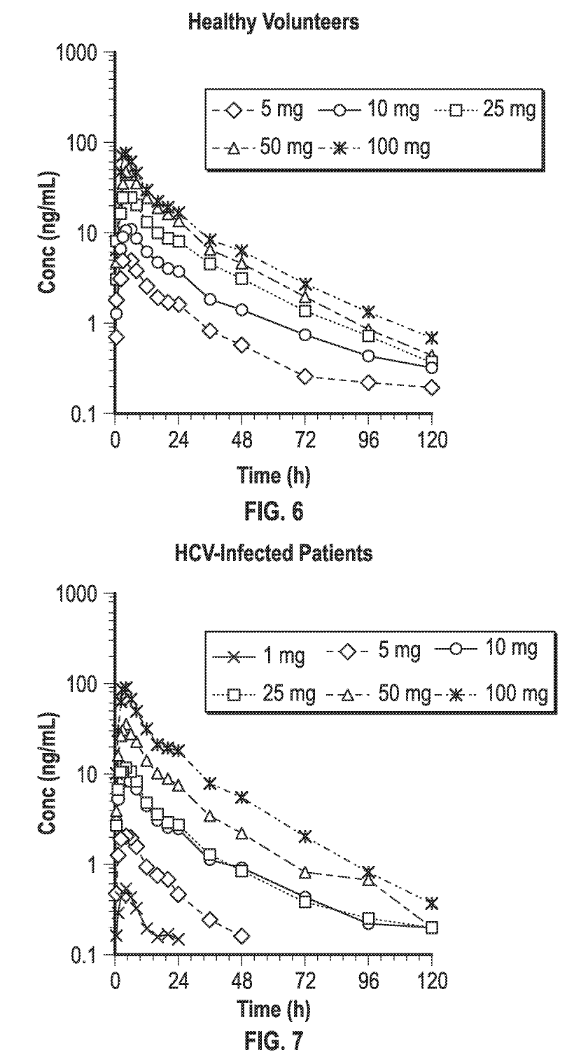

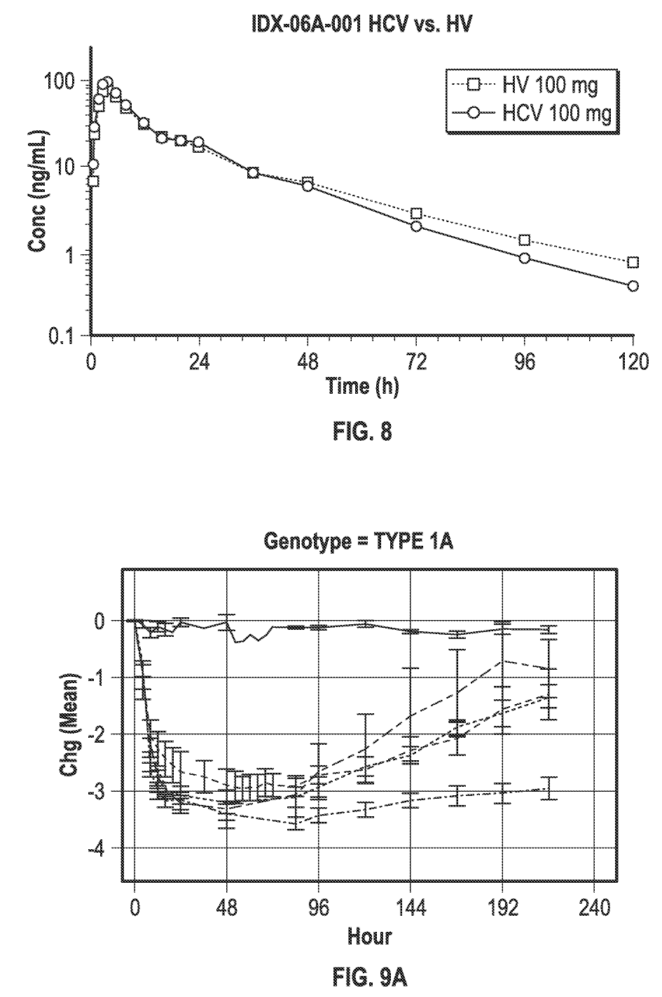

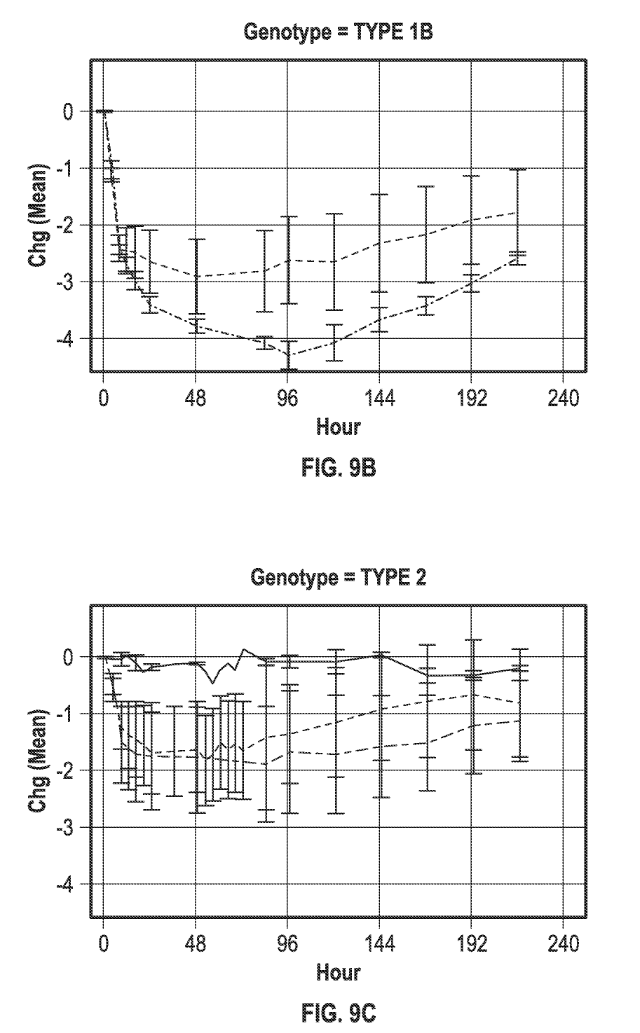

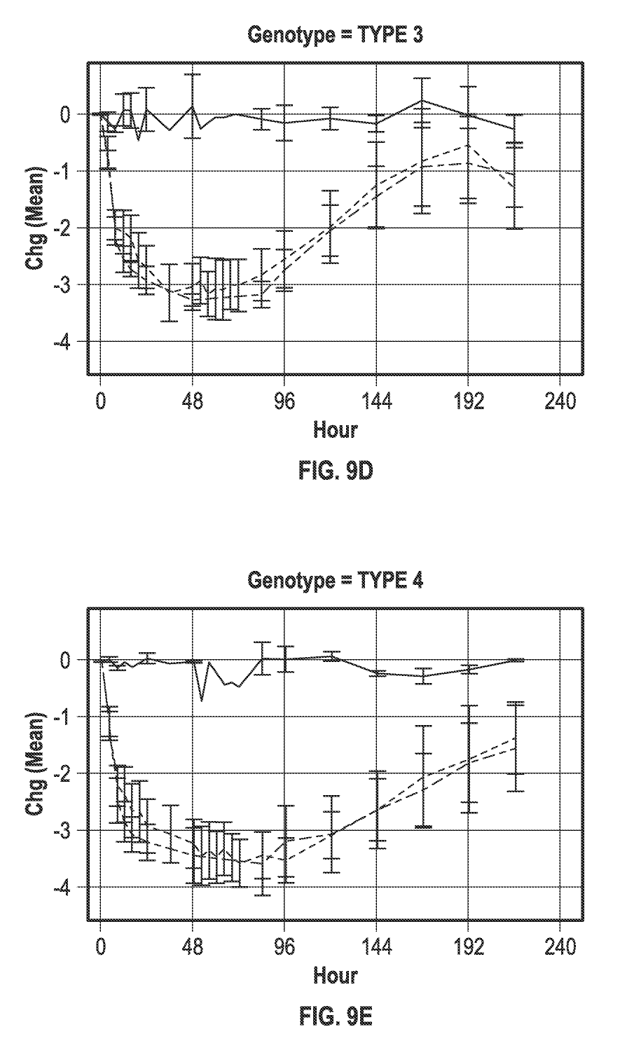

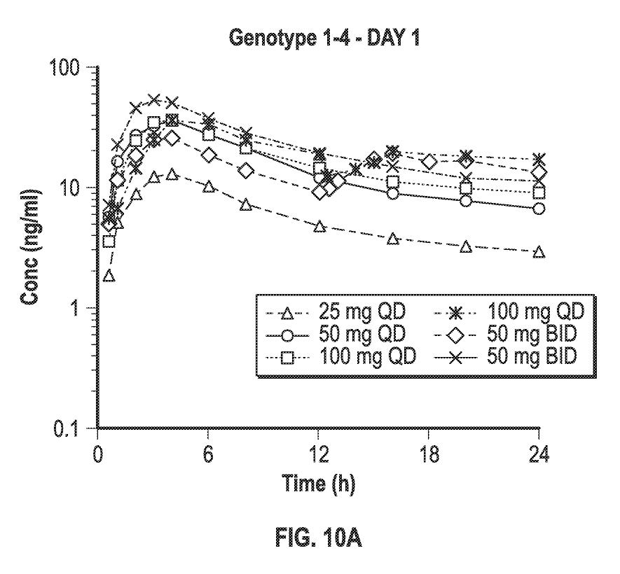

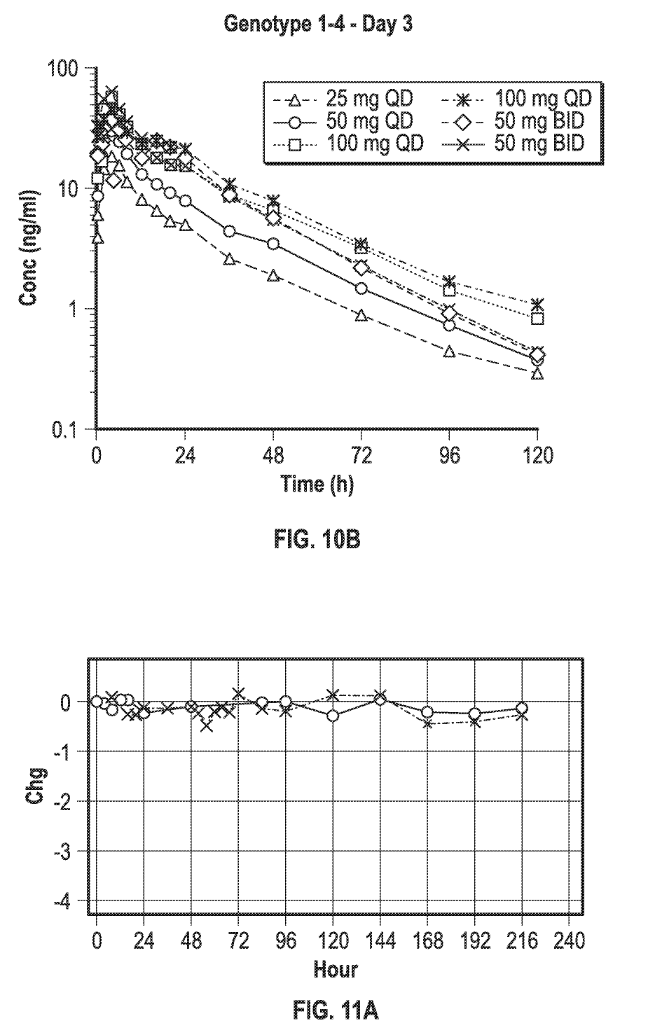

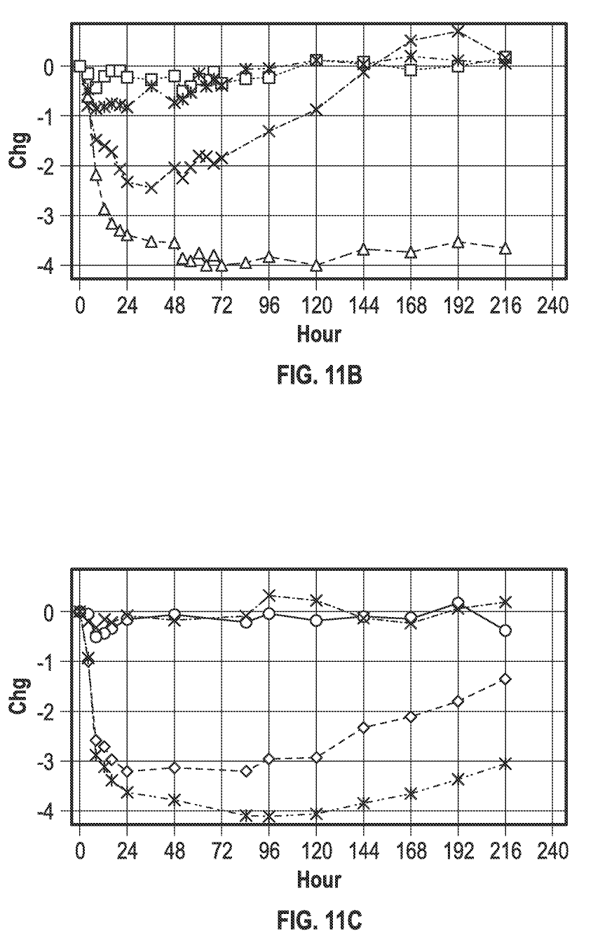

### DETAILED DESCRIPTION

To facilitate understanding of the disclosure set forth herein, a number of terms are defined below.

Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject, in one embodiment, a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The term “therapeutically effective amount” are meant to include the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician.

The term “IC₅₀” or “EC₅₀” refers an amount, concentration, or dosage of a compound that is required for 50% inhibition of a maximal response in an assay that measures such response.

The term “CC₅₀” refers an amount, concentration, or dosage of a compound that results in 50% reduction of the viability of a host. In certain embodiments, the CC₅₀ of a compound is the amount, concentration, or dosage of the compound that is required to reduce the viability of cells treated with the compound by 50%, in comparison with cells untreated with the compound.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The terms “active ingredient” and “active substance” refer to a compound, which is administered, alone or in combination with one or more pharmaceutically acceptable excipients, to a subject for treating, preventing, or ameliorating one or more symptoms of a condition, disorder, or disease. As used herein, “active ingredient” and “active substance” may be an optically active isomer or an isotopic variant of a compound described herein.

The terms “drug,” “therapeutic agent,” and “chemotherapeutic agent” refer to a compound, or a pharmaceutical composition thereof, which is administered to a subject for treating, preventing, or ameliorating one or more symptoms of a condition, disorder, or disease.

The term “hepatitis C virus” or “HCV” refers to a viral species or a variant thereof, a pathogenic strain of which causes hepatitis C. Examples of HCV include, but are not limited to, HCV genotypes 1, 2, 3, 4, 5, 6, 7, 8, 9, 10, 11, and subtype 1a, 1b, 1c, 2a, 2b, 2c, 3a, 3b, 4a, 4b, 4c, 4d, 4e, 5a, 6a, 7a, 7b, 8a, 8b, 9a, 10a, and 11a. In certain embodiments, an HCV variant is an HCV species that contains a protein substantially homologous to a native HCV protein, i.e., a protein having one or more naturally or non-naturally occurring amino acid deletions, insertions or substitutions (e.g., derivatives, homologs, and fragments), as compared to the amino acid sequence of the native protein. The amino acid sequence of a protein of an HCV variant is at least about 80% identical, at least about 90% identical, or at least about 95% identical to a native HCV protein. In certain embodiments, the HCV variant contains an NS5A protein variant.

The term “NS5A” refers to nonstructural protein 5A or a variant thereof. NS5A variants include proteins substantially homologous to a native NS5A, i.e., proteins having one or more naturally or non-naturally occurring amino acid deletions, insertions or substitutions (e.g., NS5A derivatives, homologs, and fragments), as compared to the amino acid sequence of a native NS5A. The amino acid sequence of an NS5A variant is at least about 80% identical, at least about 90% identical, or at least about 95% identical to a native NS5A.

In certain embodiments, “optically active” and “enantiomerically active” refer to a collection of molecules, which has an enantiomeric excess of no less than about 50%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, the compound comprises about 95% or more of one enantiomer and about 5% or less of the other enantiomer based on the total weight of the racemate in question.

In describing an optically active compound, the prefixes R and S are used to denote the absolute configuration of the molecule about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the molecule, R and S.

The term “isotopic variant” refers to a compound that contains an unnatural proportion of an isotope at one or more of the atoms that constitute such compounds. In certain embodiments, an “isotopic variant” of a compound contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an “isotopic variant” of a compound is in a stable form, that is, non-radioactive. In certain embodiments, an “isotopic variant” of a compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an “isotopic variant” of a compound is in an unstable form, that is, radioactive. In certain embodiments, an “isotopic variant” of a compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I), iodine-129 (¹²⁹I), and iodine-131 (¹³¹I). It will be understood that, in a compound as provided herein, any hydrogen can be ²H, for example, or any carbon can be ¹³C, as example, or any nitrogen can be ¹⁵N, as example, and any oxygen can be ¹⁸O, where feasible according to the judgment of one of skill. In certain embodiments, an “isotopic variant” of a compound contains unnatural proportions of deuterium.

The term “solvate” refers to a complex or aggregate formed by one or more molecules of a solute, e.g., a compound provided herein, and one or more molecules of a solvent, which present in stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The phrase “an isotopic variant thereof; or a pharmaceutically acceptable salt or solvate thereof” has the same meaning as the phrase “an isotopic variant of the compound referenced therein; or a pharmaceutically acceptable salt or solvate of the compound referenced therein; or a pharmaceutically acceptable salt or solvate of an isotopic variant of the compound referenced therein.”

### Compound

HCV has a single positive-stranded RNA genome having about 9.6 kb in length that encodes a large polyprotein having about 3010 amino acids. This precursor polyprotein is then processed into a range of structural proteins, including core protein, C, and envelope glycoproteins, E1 and E2; and non-structural proteins, including NS2, NS3, NS4A, NS4B, NS5A, and NS5B, by host signal peptidases and two viral proteases, NS2-3 and NS3. The nonstructural protein 5A (NS5A) is a multifunctional protein essential for HCV replication. Because of its vital role in viral replication, HCV NS5A protein has been actively pursued as a drug target for developing anti-HCV therapy.

In one embodiment, provided herein is [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester (“the Compound”), having the structure of Formula I:

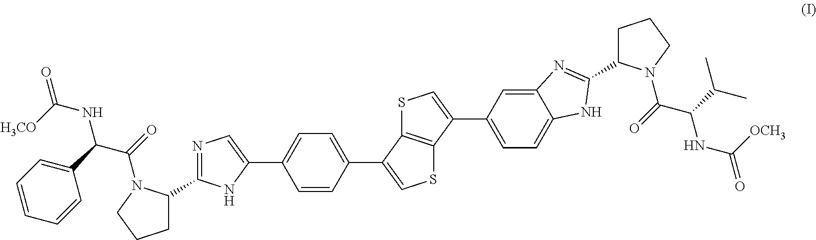

or an isotopic variant thereof, or a pharmaceutically acceptable salt or solvate thereof.

The Compound is a nonstructural protein 5A (NS5A) inhibitor. See U.S. Pat. App. Pub. Nos. US 2011/0150827 and US 2012/0252721, the disclosure of each of which is incorporated herein by reference in its entirety. The Compound is a potent and pan-genotypic inhibitor of HCV replication in vitro, with EC₅₀ values ranging from 2 to 24 pM against HCV genotypes 1a, 1b, 2a, 3a, 4a, and 5a. Id.

The Compound can be prepared according to the methods described in U.S. Pat. App. Pub. No. US 2011/0150827. The Compound can be also synthesized according to other methods apparent to those of skill in the art based upon the teaching herein.

In certain embodiments, the compound used in the methods provided herein is the Compound. In certain embodiments, the compound used in the methods provided herein is an isotopic variant of the Compound, or a pharmaceutically acceptable salt or solvate thereof. In certain embodiments, the compound used in the methods provided herein is an isotopic variant of the Compound.

In certain embodiments, the compound used in the methods provided herein is a pharmaceutically acceptable salt of the Compound, which includes, but is not limited to, acetate, adipate, alginate, aspartate, benzoate, benzenesulfonate (besylate), bisulfate, butyrate, citrate, camphorate, camphorsulfonate, cyclopentanepropionate, digluconate, dodecylsulfate, 1,2-ethanedisulfonate (edisylate), ethanesulfonate (esylate), formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate (mesylate), 2-naphthalenesulfonate (napsylate), nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate, or undecanoate salts.

As used herein, the Compound is intended to encompass all possible stereoisomers, unless a particular stereochemistry is specified. Where structural isomers of the Compound are interconvertible via a low energy barrier, the Compound may exist as a single tautomer or a mixture of tautomers. This can take the form of proton tautomerism in the compound that contains, e.g., an imidazolyl or benzimidazolyl group; or so-called valence tautomerism in the compound that contain an aromatic moiety.

### Pharmaceutical Compositions

In one embodiment, provided herein is a pharmaceutical composition comprising the Compound or an isotopic variant, or a pharmaceutically acceptable salt o solvate thereof; and a pharmaceutically acceptable excipient.

Suitable excipients are well known to those skilled in the art, and non-limiting examples of suitable excipients are provided herein. Whether a particular excipient is suitable for incorporation into a pharmaceutical composition or dosage form depends on a variety of factors well known in the art, including, but not limited to, the method of administration. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, provided herein are pharmaceutical compositions and dosage forms that contain little, if any, lactose, or other mono- or di-saccharides. As used herein, the term “lactose-free” means that the amount of lactose present, if any, is insufficient to substantially increase the degradation rate of an active ingredient. In one embodiment, lactose-free compositions comprise an active ingredient provided herein, a binder/filler, and a lubricant. In another embodiment, lactose-free dosage forms comprise an active ingredient, microcrystalline cellulose, pre-gelatinized starch, and magnesium stearate.

The pharmaceutical compositions provided herein can be formulated in various dosage forms for oral, parenteral, and topical administration. The pharmaceutical compositions can also be formulated as modified release dosage forms, including delayed-, extended-, prolonged-, sustained-, pulsatile-, controlled-, accelerated-, fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Delivery Technology, 2nd ed.; Rathbone et al., Eds.; Marcel Dekker, Inc.: New York, N.Y., 2008).

In one embodiment, provided herein is a pharmaceutical composition in a dosage form for oral administration, which comprises the Compound or an isotopic variant thereof, or a pharmaceutically acceptable salt or solvate thereof; and a pharmaceutically acceptable excipient.

In another embodiment, provided herein is a pharmaceutical composition in a dosage form for parenteral administration, which comprises the Compound or an isotopic variant thereof, or a pharmaceutically acceptable salt or solvate thereof; and a pharmaceutically acceptable excipient.

In yet another embodiment, provided herein is a pharmaceutical composition in a dosage form for topical administration, which comprises the Compound or an isotopic variant thereof, or a pharmaceutically acceptable salt or solvate thereof; and a pharmaceutically acceptable excipient.

In one embodiment, provided herein is a suspension formulation for oral administration, comprising spray dried dispersion of the Compound or an isotopic variant thereof, or a pharmaceutically acceptable salt or solvate thereof; and a pharmaceutically acceptable excipient.

In certain embodiments, the suspension formulation of the Compound comprises spray dried dispersion of the Compound or an isotopic variant thereof, or a pharmaceutically acceptable salt or solvate thereof; a solvent, and a flavoring agent, an emulsifier, or a thickener.

In certain embodiments, the suspension formulation of the Compound comprises spray dried dispersion of the Compound or an isotopic variant thereof, or a pharmaceutically acceptable salt or solvate thereof; a solvent, a flavoring agent, and an emulsifier.

In certain embodiments, the suspension formulation of the Compound comprises spray dried dispersion of the Compound or an isotopic variant thereof, or a pharmaceutically acceptable salt or solvate thereof; a solvent, a flavoring agent, an emulsifier, and a thickener.

In certain embodiments, the suspension formulation of the Compound comprises spray dried dispersion of the Compound or an isotopic variant thereof, or a pharmaceutically acceptable salt or solvate thereof; water, and expresso flavoring syrup, methylcellulose, or colloidal silica.

In certain embodiments, the suspension formulation of the Compound comprises spray dried dispersion of the Compound or an isotopic variant thereof, or a pharmaceutically acceptable salt or solvate thereof; water, expresso flavoring syrup, and methylcellulose.

In certain embodiments, the suspension formulation of the Compound comprises spray dried dispersion of the Compound or an isotopic variant thereof, or a pharmaceutically acceptable salt or solvate thereof; water, expresso flavoring syrup, methylcellulose, and colloidal silica.

In certain embodiments, the suspension formulation of the Compound comprises spray dried dispersion of the Compound or an isotopic variant thereof, or a pharmaceutically acceptable salt or solvate thereof; water, and expresso flavoring syrup, METHOCEL® A4M premium, or CAR-O-SIL® M5P.

In certain embodiments, the suspension formulation of the Compound comprises spray dried dispersion of the Compound or an isotopic variant thereof, or a pharmaceutically acceptable salt or solvate thereof; water, expresso flavoring syrup, and METHOCEL® A4M premium.

In certain embodiments, the suspension formulation of the Compound comprises spray dried dispersion of the Compound or an isotopic variant thereof, or a pharmaceutically acceptable salt or solvate thereof; water, expresso flavoring syrup, METHOCEL® A4M premium, and CAR-O-SIL® M5P.

In certain embodiments, the suspension formulation of the Compound comprises spray dried dispersion of the Compound or an isotopic variant thereof, or a pharmaceutically acceptable salt or solvate thereof; and a solvent.

In certain embodiments, the suspension formulation of the Compound comprises spray dried dispersion of the Compound or an isotopic variant thereof, or a pharmaceutically acceptable salt or solvate thereof; and water.

In certain embodiments, the suspension formulation of the Compound comprises spray dried dispersion of the Compound or an isotopic variant thereof, or a pharmaceutically acceptable salt or solvate thereof; and a flavoring agent.

In certain embodiments, the suspension formulation of the Compound comprises spray dried dispersion of the Compound or an isotopic variant thereof, or a pharmaceutically acceptable salt or solvate thereof; and expresso flavoring syrup.

In certain embodiments, the suspension formulation of the Compound comprises spray dried dispersion of the Compound or an isotopic variant thereof, or a pharmaceutically acceptable salt or solvate thereof; and an emulsifier.

In certain embodiments, the suspension formulation of the Compound comprises spray dried dispersion of the Compound or an isotopic variant thereof, or a pharmaceutically acceptable salt or solvate thereof; and methylcellulose.

In certain embodiments, the suspension formulation of the Compound comprises spray dried dispersion of the Compound or an isotopic variant thereof, or a pharmaceutically acceptable salt or solvate thereof; and METHOCEL® A4M premium.

In certain embodiments, the suspension formulation of the Compound comprises spray dried dispersion of the Compound or an isotopic variant thereof, or a pharmaceutically acceptable salt or solvate thereof; and a thickener.

In certain embodiments, the suspension formulation of the Compound comprises spray dried dispersion of the Compound or an isotopic variant thereof, or a pharmaceutically acceptable salt or solvate thereof; and colloidal silica.

In certain embodiments, the suspension formulation of the Compound comprises spray dried dispersion of the Compound or an isotopic variant thereof, or a pharmaceutically acceptable salt or solvate thereof; and CAR-O-SIL® M5P.

The pharmaceutical compositions provided herein can be provided in a unit-dosage form or multiple-dosage form. A unit-dosage form, as used herein, refers to physically discrete a unit suitable for administration to a human and animal subject, and packaged individually as is known in the art. Each unit-dose contains a predetermined quantity of an active ingredient(s) sufficient to produce the desired therapeutic effect, in association with the required pharmaceutical carriers or excipients. Examples of a unit-dosage form include an ampoule, syringe, and individually packaged tablet and capsule. For example, a 100 mg unit dose contains about 100 mg of an active ingredient in a packaged tablet or capsule. A unit-dosage form may be administered in fractions or multiples thereof. A multiple-dosage form is a plurality of identical unit-dosage forms packaged in a single container to be administered in segregated unit-dosage form. Examples of a multiple-dosage form include a vial, bottle of tablets or capsules, or bottle of pints or gallons.

The pharmaceutical compositions provided herein can be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

### A. Oral Administration

The pharmaceutical compositions provided herein for oral administration can be provided in solid, semisolid, or liquid dosage forms for oral administration. As used herein, oral administration also includes buccal, lingual, and sublingual administration. Suitable oral dosage forms include, but are not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, and syrups. In addition to the active ingredient(s), the pharmaceutical compositions can contain one or more pharmaceutically acceptable carriers or excipients, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The amount of a binder or filler in the pharmaceutical compositions provided herein varies upon the type of formulation, and is readily discernible to those of ordinary skill in the art. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets. The amount of a diluent in the pharmaceutical compositions provided herein varies upon the type of formulation, and is readily discernible to those of ordinary skill in the art.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of a disintegrant in the pharmaceutical compositions provided herein varies upon the type of formulation, and is readily discernible to those of ordinary skill in the art. The amount of a disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include, but are not limited to, colloidal silicon dioxide, CAB-O-SIL® (Cabot Co. of Boston, Mass.), and asbestos-free talc. Suitable coloring agents include, but are not limited to, any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Suitable flavoring agents include, but are not limited to, natural flavors extracted from plants, such as fruits, and synthetic blends of compounds which produce a pleasant taste sensation, such as peppermint and methyl salicylate. Suitable sweetening agents include, but are not limited to, sucrose, lactose, mannitol, syrups, glycerin, and artificial sweeteners, such as saccharin and aspartame. Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suitable suspending and dispersing agents include, but are not limited to, sodium carboxymethylcellulose, pectin, tragacanth, Veegum, acacia, sodium carbomethylcellulose, hydroxypropyl methylcellulose, and polyvinylpyrrolidone. Suitable preservatives include, but are not limited to, glycerin, methyl and propylparaben, benzoic add, sodium benzoate and alcohol. Suitable wetting agents include, but are not limited to, propylene glycol monostearate, sorbitan monooleate, diethylene glycol monolaurate, and polyoxyethylene lauryl ether. Suitable solvents include, but are not limited to, glycerin, sorbitol, ethyl alcohol, and syrup. Suitable non-aqueous liquids utilized in emulsions include, but are not limited to, mineral oil and cottonseed oil. Suitable organic acids include, but are not limited to, citric and tartaric acid. Suitable sources of carbon dioxide include, but are not limited to, sodium bicarbonate and sodium carbonate.

It should be understood that many carriers and excipients may serve a plurality of functions, even within the same formulation.

The pharmaceutical compositions provided herein for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms can be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein for oral administration can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions provided herein for oral administration can be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquid or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations can further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration can be also provided in the forms of liposomes, micelles, microspheres, or nanosystems. Micellar dosage forms can be prepared as described in U.S. Pat. No. 6,350,458.

The pharmaceutical compositions provided herein for oral administration can be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein for oral administration can be formulated as immediate or modified release dosage forms, including delayed-, sustained, pulsed-, controlled, targeted-, and programmed-release forms.

### B. Parenteral Administration

The pharmaceutical compositions provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, intravesical, and subcutaneous administration.

The pharmaceutical compositions provided herein for parenteral administration can be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration can include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Suitable water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents are those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

When the pharmaceutical compositions provided herein are formulated for multiple dosage administration, the multiple dosage parenteral formulations must contain an antimicrobial agent at bacteriostatic or fungistatic concentrations. All parenteral formulations must be sterile, as known and practiced in the art.

In one embodiment, the pharmaceutical compositions for parenteral administration are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein for parenteral administration can be formulated as immediate or modified release dosage forms, including delayed-, sustained, pulsed-, controlled, targeted-, and programmed-release forms.

The pharmaceutical compositions provided herein for parenteral administration can be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include, but are not limited to, polymethylmethacrylate, polybutyl-methacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers, such as hydrogels of esters of acrylic and methacrylic acid, collagen, cross-linked polyvinyl alcohol, and cross-linked partially hydrolyzed polyvinyl acetate.

Suitable outer polymeric membranes include but are not limited to, polyethylene, polypropylene, ethylene/propylene copolymers, ethylene/ethyl acrylate copolymers, ethylene/vinyl acetate copolymers, silicone rubbers, polydimethyl siloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### C. Topical Administration

The pharmaceutical compositions provided herein can be administered topically to the skin, orifices, or mucosa. The topical administration, as used herein, includes (intra)dermal, conjunctival, intracorneal, intraocular, ophthalmic, auricular, transdermal, nasal, vaginal, urethral, respiratory, and rectal administration.

The pharmaceutical compositions provided herein can be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, and dermal patches. The topical formulation of the pharmaceutical compositions provided herein can also comprise liposomes, micelles, microspheres, nanosystems, and mixtures thereof.

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The pharmaceutical compositions can also be administered topically by electroporation, iontophoresis, phonophoresis, sonophoresis, or microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions provided herein can be provided in the forms of ointments, creams, and gels. Suitable ointment vehicles include oleaginous or hydrocarbon vehicles, including lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Suitable cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include, but are not limited to, crosslinked acrylic acid polymers, such as carbomers, carboxypolyalkylenes, and CARBOPOL®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions provided herein can be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions provided herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, and hydrogels, such as polyvinyl alcohol, hydroxyethyl methacrylate, and polyacrylic acid. Combinations of the various vehicles can also be used. Rectal and vaginal suppositories may be prepared by compressing or molding. The typical weight of a rectal and vaginal suppository is about 2 to about 3 g.

The pharmaceutical compositions provided herein can be administered ophthalmically in the forms of solutions, suspensions, ointments, emulsions, gel-forming solutions, powders for solutions, gels, ocular inserts, and implants.

The pharmaceutical compositions provided herein can be administered intranasally or by inhalation to the respiratory tract. The pharmaceutical compositions can be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions can also be provided as a dry powder for insufflation, alone or in combination with an inert carrier such as lactose or phospholipids; and nasal drops. For intranasal use, the powder can comprise a bioadhesive agent, including chitosan or cyclodextrin.

Solutions or suspensions for use in a pressurized container, pump, spray, atomizer, or nebulizer can be formulated to contain ethanol, aqueous ethanol, or a suitable alternative agent for dispersing, solubilizing, or extending release of the active ingredient provided herein; a propellant as solvent; and/or a surfactant, such as sorbitan trioleate, oleic acid, or an oligolactic acid.

The pharmaceutical compositions provided herein can be micronized to a size suitable for delivery by inhalation, such as about 50 micrometers or less, or about 10 micrometers or less. Particles of such sizes can be prepared using a comminuting method known to those skilled in the art, such as spiral jet milling, fluid bed jet milling, supercritical fluid processing to form nanoparticles, high pressure homogenization, or spray drying.

Capsules, blisters, and cartridges for use in an inhaler or insufflator can be formulated to contain a powder mix of the pharmaceutical compositions provided herein; a suitable powder base, such as lactose or starch; and a performance modifier, such as 1-leucine, mannitol, or magnesium stearate. The lactose may be anhydrous or in the form of the monohydrate. Other suitable excipients or carriers include, but are not limited to, dextran, glucose, maltose, sorbitol, xylitol, fructose, sucrose, and trehalose. The pharmaceutical compositions provided herein for inhaled/intranasal administration can further comprise a suitable flavor, such as menthol and levomenthol; and/or sweeteners, such as saccharin and saccharin sodium.

The pharmaceutical compositions provided herein for topical administration can be formulated to be immediate release or modified release, including delayed-, sustained-, pulsed-, controlled-, targeted, and programmed release.

### D. Modified Release

The pharmaceutical compositions provided herein can be formulated as a modified release dosage form. As used herein, the term “modified release” refers to a dosage form in which the rate or place of release of the active ingredient(s) is different from that of an immediate dosage form when administered by the same route. Modified release dosage forms include, but are not limited to, delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

Examples of modified release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,958,458; 5,972,891; 5,980,945; 5,993,855; 6,045,830; 6,087,324; 6,113,943; 6,197,350; 6,248,363; 6,264,970; 6,267,981; 6,270,798; 6,375,987; 6,376,461; 6,419,961; 6,589,548; 6,613,358; 6,623,756; 6,699,500; 6,793,936; 6,827,947; 6,902,742; 6,958,161; 7,255,876; 7,416,738; 7,427,414; 7,485,322; Bussemer et al., Crit. Rev. Ther. Drug Carrier Syst. 2001, 18, 433-458; Modified-Release Drug Delivery Technology, 2nd ed.; Rathbone et al., Eds.; Marcel Dekker AG: 2005; Maroni et al., Expert. Opin. Drug Deliv. 2005, 2, 855-871; Shi et al., Expert Opin. Drug Deliv. 2005, 2, 1039-1058; Polymers in Drug Delivery; Ijeoma et al., Eds.; CRC Press LLC: Boca Raton, Fla., 2006; Badawy et al., J. Pharm. Sci. 2007, 9, 948-959; Modified-Release Drug Delivery Technology, supra; Conway, Recent Pat. Drug Deliv. Formul. 2008, 2, 1-8; Gazzaniga et al., Eur. J. Pharm. Biopharm. 2008, 68, 11-18; Nagarwal et al., Curr. Drug Deliv. 2008, 5, 282-289; Gallardo et al., Pharm. Dev. Technol. 2008, 13, 413-423; Chrzanowski, AAPS PharmSciTech. 2008, 9, 635-638; Chrzanowski, AAPS PharmSciTech. 2008, 9, 639-645; Kalantzi et al., Recent Pat. Drug Deliv. Formul. 2009, 3, 49-63; Saigal et al., Recent Pat. Drug Deliv. Formul. 2009, 3, 64-70; and Roy et al., J. Control Release 2009, 134, 74-80.

### 1. Matrix Controlled Release Devices

The pharmaceutical compositions provided herein in a modified release dosage form can be fabricated using a matrix controlled release device known to those skilled in the art. See, Takada et al. in Encyclopedia of Controlled Drug Delivery; Mathiowitz Ed.; Wiley: 1999; Vol 2.

In certain embodiments, the pharmaceutical compositions provided herein in a modified release dosage form is formulated using an erodible matrix device, which is water-swellable, erodible, or soluble polymers, including, but not limited to, synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethyl hydroxyethyl cellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methyl methacrylate, ethyl methacrylate, ethylacrylate, (2-dimethylaminoethyl)methacrylate, and (trimethylaminoethyl)methacrylate chloride.

In certain embodiments, the pharmaceutical compositions provided herein are formulated with a non-erodible matrix device. The active ingredient(s) is dissolved or dispersed in an inert matrix and is released primarily by diffusion through the inert matrix once administered. Materials suitable for use as a non-erodible matrix device include, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinyl acetate copolymers, ethylene/propylene copolymers, ethylene/ethyl acrylate copolymers, vinyl chloride copolymers with vinyl acetate, vinylidene chloride, ethylene and propylene, ionomer polyethylene terephthalate, butyl rubbers, epichlorohydrin rubbers, ethylene/vinyl alcohol copolymer, ethylene/vinyl acetate/vinyl alcohol terpolymer, ethylene/vinyloxyethanol copolymer, polyvinyl chloride, plasticized nylon, plasticized polyethylene terephthalate, natural rubber, silicone rubbers, polydimethylsiloxanes, and silicone carbonate copolymers; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

The pharmaceutical compositions provided herein in a modified release dosage form can be prepared by methods known to those skilled in the art, including direct compression, dry or wet granulation followed by compression, and melt-granulation followed by compression.

### 2. Osmotic Controlled Release Devices

The pharmaceutical compositions provided herein in a modified release dosage form can be fabricated using an osmotic controlled release device, including, but not limited to, one-chamber system, two-chamber system, asymmetric membrane technology (AMT), and extruding core system (ECS). In general, such devices have at least two components: (a) a core which contains an active ingredient; and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents is water-swellable hydrophilic polymers, which are also referred to as “osmopolymers” and “hydrogels.” Suitable water-swellable hydrophilic polymers as osmotic agents include, but are not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents is osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates can be employed to influence how rapidly the active ingredient(s) is initially delivered from the dosage form. For example, amorphous sugars, such as MANNOGEM™ EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core can also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane can also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane can be formed post-coating by mechanical or laser drilling. Delivery port(s) can also be formed in situ by erosion of a plug of water-soluble material or by rupture of a thinner portion of the membrane over an indentation in the core. In addition, delivery ports can be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form can further comprise additional conventional excipients or carriers as described herein to promote performance or processing of the formulation.

The osmotic controlled-release dosage forms can be prepared according to conventional methods and techniques known to those skilled in the art. See, Remington: The Science and Practice of Pharmacy, supra; Santus and Baker, J. Controlled Release 1995, 35, 1-21; Verma et al., Drug Development and Industrial Pharmacy 2000, 26, 695-708; and Verma et al., J. Controlled Release 2002, 79, 7-27.

In certain embodiments, the pharmaceutical compositions provided herein are formulated as AMT controlled-release dosage form, which comprises an asymmetric osmotic membrane that coats a core comprising the active ingredient(s) and other pharmaceutically acceptable excipients or carriers. See, U.S. Pat. No. 5,612,059 and International Pat. App. Pub. No. WO 2002/17918. The AMT controlled-release dosage forms can be prepared according to conventional methods and techniques known to those skilled in the art, including direct compression, dry granulation, wet granulation, and a dip-coating method.

In certain embodiments, the pharmaceutical compositions provided herein are formulated as ESC controlled-release dosage form, which comprises an osmotic membrane that coats a core comprising the active ingredient(s), a hydroxylethyl cellulose, and other pharmaceutically acceptable excipients or carriers.

### 3. Multiparticulate Controlled Release Devices

The pharmaceutical compositions provided herein in a modified release dosage form can be fabricated as a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates can be made by the processes known to those skilled in the art, including wet- and dry-granulation, extrusion/spheronization, roller-compaction, melt-congealing, and by spray-coating seed cores. See, for example, Multiparticulate Oral Drug Delivery; Ghebre-Sellassie Ed.; Marcel Dekker: 1994; and Pharmaceutical Pelletization Technology; Ghebre-Sellassie Ed.; Marcel Dekker: 1989.

Other excipients or carriers as described herein can be blended with the pharmaceutical compositions to aid in processing and forming the multiparticulates. The resulting particles can themselves constitute the multiparticulate device or can be coated by various film-forming materials, such as enteric polymers, water-swellable, and water-soluble polymers. The multiparticulates can be further processed as a capsule or a tablet.

### 4. Targeted Delivery

The pharmaceutical compositions provided herein can also be formulated to be targeted to a particular tissue, receptor, or other area of the body of the subject to be treated, including liposome-, resealed erythrocyte-, and antibody-based delivery systems. Examples include, but are not limited to, those disclosed in U.S. Pat. Nos. 5,709,874; 5,759,542; 5,840,674; 5,900,252; 5,972,366; 5,985,307; 6,004,534; 6,039,975; 6,048,736; 6,060,082; 6,071,495; 6,120,751; 6,131,570; 6,139,865; 6,253,872; 6,271,359; 6,274,552; 6,316,652; and 7,169,410.

### Methods of Use

In one embodiment, provided herein is a method for treating or preventing a hepatitis C viral infection in a subject, which comprises administering to the subject a therapeutically effective amount of [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester (“the Compound”), or an isotopic variant thereof, or a pharmaceutically acceptable salt or solvate thereof.

In another embodiment, provided herein is a method for treating, preventing, or ameliorating one or more symptoms of a liver disease or disorder associated with an HCV infection in a subject, comprising administering to the subject a therapeutically effective amount of the Compound or an isotopic variant thereof, or a pharmaceutically acceptable salt or solvate thereof. In certain embodiments, the liver disease or disorder associated with an HCV infection is chronic hepatitis, cirrhosis, hepatocarcinoma, or extra hepatic manifestation.

In certain embodiments, the therapeutically effective amount of the Compound or an isotopic variant thereof, or a pharmaceutically acceptable salt or solvate thereof, is at least about 1 mg per day, at least about 5 mg per day, at least about 10 mg per day, or at least about 20 mg per day. In certain embodiments, the therapeutically effective amount of the Compound or an isotopic variant thereof, or a pharmaceutically acceptable salt or solvate thereof, is at least about 1 mg per day. In certain embodiments, the therapeutically effective amount of the Compound or an isotopic variant thereof, or a pharmaceutically acceptable salt or solvate thereof, is at least about 5 mg per day. In certain embodiments, the therapeutically effective amount of the Compound or an isotopic variant thereof, or a pharmaceutically acceptable salt or solvate thereof, is at least about 10 mg per day. In certain embodiments, the therapeutically effective amount of the Compound or an isotopic variant thereof, or a pharmaceutically acceptable salt or solvate thereof, is at least about 20 mg per day.

In certain embodiments, the therapeutically effective amount of the Compound or an isotopic variant thereof, or a pharmaceutically acceptable salt or solvate thereof, is ranging from about 1 to about 1,000 mg per day, from about 1 to about 500 mg per day, from about 5 to about 500 mg per day, from about 5 to about 200 mg per day, from about 5 to about 100 mg per day, or from about 10 to about 100 mg per day. In certain embodiments, the therapeutically effective amount of the Compound or an isotopic variant thereof, or a pharmaceutically acceptable salt or solvate thereof, is ranging from about 1 to about 1,000 mg per day. In certain embodiments, the therapeutically effective amount of the Compound or an isotopic variant thereof, or a pharmaceutically acceptable salt or solvate thereof, is ranging from about 1 to about 500 mg per day. In certain embodiments, the therapeutically effective amount of the Compound or an isotopic variant thereof, or a pharmaceutically acceptable salt or solvate thereof, is ranging from about 5 to about 500 mg per day. In certain embodiments, the therapeutically effective amount of the Compound or an isotopic variant thereof, or a pharmaceutically acceptable salt or solvate thereof, is ranging from about 5 to about 200 mg per day. In certain embodiments, the therapeutically effective amount of the Compound or an isotopic variant thereof, or a pharmaceutically acceptable salt or solvate thereof, is ranging from about 5 to about 100 mg per day. In certain embodiments, the therapeutically effective amount of the Compound or an isotopic variant thereof, or a pharmaceutically acceptable salt or solvate thereof, is ranging from about 10 to about 100 mg per day.

In certain embodiments, the therapeutically effective amount of the Compound or an isotopic variant thereof, or a pharmaceutically acceptable salt or solvate thereof, is about 1 mg per day, about 2 mg per day, about 5 mg per day, about 10 mg per day, about 25 mg per day, about 50 mg per day, about 100 mg per day, about 200 mg per day, about 500 mg per day, or about 1,000 mg per day. In certain embodiments, the therapeutically effective amount of the Compound or an isotopic variant thereof, or a pharmaceutically acceptable salt or solvate thereof, is about 1 mg per day. In certain embodiments, the therapeutically effective amount of the Compound or an isotopic variant thereof, or a pharmaceutically acceptable salt or solvate thereof, is about 2 mg per day. In certain embodiments, the therapeutically effective amount of the Compound or an isotopic variant thereof, or a pharmaceutically acceptable salt or solvate thereof, is about 5 mg per day. In certain embodiments, the therapeutically effective amount of the Compound or an isotopic variant thereof, or a pharmaceutically acceptable salt or solvate thereof, is about 10 mg per day. In certain embodiments, the therapeutically effective amount of the Compound or an isotopic variant thereof, or a pharmaceutically acceptable salt or solvate thereof, is about 25 mg per day. In certain embodiments, the therapeutically effective amount of the Compound or an isotopic variant thereof, or a pharmaceutically acceptable salt or solvate thereof, is about 50 mg per day. In certain embodiments, the therapeutically effective amount of the Compound or an isotopic variant thereof, or a pharmaceutically acceptable salt or solvate thereof, is about 100 mg per day. In certain embodiments, the therapeutically effective amount of the Compound or an isotopic variant thereof, or a pharmaceutically acceptable salt or solvate thereof, is about 200 mg per day. In certain embodiments, the therapeutically effective amount of the Compound or an isotopic variant thereof, or a pharmaceutically acceptable salt or solvate thereof, is about 500 mg per day. In certain embodiments, the therapeutically effective amount of the Compound or an isotopic variant thereof, or a pharmaceutically acceptable salt or solvate thereof, is about 1,000 mg per day.

In certain embodiments, the therapeutically effective amount of the Compound or an isotopic variant thereof, or a pharmaceutically acceptable salt or solvate thereof, is about 1 mg per day, about 5 mg per day, about 10 mg per day, about 25 mg per day, about 50 mg per day, or about 100 mg per day.

In certain embodiments, the therapeutically effective amount of the Compound or an isotopic variant thereof, or a pharmaceutically acceptable salt or solvate thereof, is ranging from about 0.02 to about 20 mg/kg/day, from about 0.1 to about 10 mg/kg/day, from about 0.1 to about 5 mg/kg/day, or from about 0.2 to about 2 mg/kg/day. In certain embodiments, the therapeutically effective amount of the Compound or an isotopic variant thereof, or a pharmaceutically acceptable salt or solvate thereof, is ranging from about 0.02 to about 20 mg/kg/day. In certain embodiments, the therapeutically effective amount of the Compound or an isotopic variant thereof, or a pharmaceutically acceptable salt or solvate thereof, is ranging from about 0.1 to about 10 mg/kg/day. In certain embodiments, the therapeutically effective amount of the Compound or an isotopic variant thereof, or a pharmaceutically acceptable salt or solvate thereof, is ranging from about 0.1 to about 5 mg/kg/day. In certain embodiments, the therapeutically effective amount of the Compound or an isotopic variant thereof, or a pharmaceutically acceptable salt or solvate thereof, is ranging from about 0.2 to about 2 mg/kg/day.

In certain embodiments, the therapeutically effective amount of the Compound or an isotopic variant thereof, or a pharmaceutically acceptable salt or solvate thereof, is about 0.02 mg/kg/day, about 0.1 mg/kg/day, about 0.2 mg/kg/day, about 0.5 mg/kg/day, about 1 mg/kg/day, or about 2 mg/kg/day. In certain embodiments, the therapeutically effective amount of the Compound or an isotopic variant thereof, or a pharmaceutically acceptable salt or solvate thereof, is about 0.02 mg/kg/day. In certain embodiments, the therapeutically effective amount of the Compound or an isotopic variant thereof, or a pharmaceutically acceptable salt or solvate thereof, is about 0.1 mg/kg/day. In certain embodiments, the therapeutically effective amount of the Compound or an isotopic variant thereof, or a pharmaceutically acceptable salt or solvate thereof, is about 0.2 mg/kg/day. In certain embodiments, the therapeutically effective amount of the Compound or an isotopic variant thereof, or a pharmaceutically acceptable salt or solvate thereof, is about 0.5 mg/kg/day. In certain embodiments, the therapeutically effective amount of the Compound or an isotopic variant thereof, or a pharmaceutically acceptable salt or solvate thereof, is about 1 mg/kg/day. In certain embodiments, the therapeutically effective amount of the Compound or an isotopic variant thereof, or a pharmaceutically acceptable salt or solvate thereof, is about 2 mg/kg/day.

In certain embodiments, the hepatitis C virus (HCV) is drug-resistant.

Thus, in one embodiment, provided herein is a method for treating or preventing a drug-resistant hepatitis C viral infection in a subject, which comprises administering to the subject a therapeutically effective amount of the Compound or an isotopic variant thereof, or a pharmaceutically acceptable salt or solvate thereof, wherein the therapeutically effective amount is as defined herein.

In another embodiment, provided herein is a method for treating, preventing, or ameliorating one or more symptoms of a liver disease or disorder associated with a drug-resistant HCV infection in a subject, comprising administering to the subject a therapeutically effective amount of the Compound or an isotopic variant thereof, or a pharmaceutically acceptable salt or solvate thereof, wherein the therapeutically effective amount is as defined herein. In certain embodiments, the liver disease or disorder associated with a drug-resistant HCV infection is chronic hepatitis, cirrhosis, hepatocarcinoma, or extra hepatic manifestation.

In certain embodiments, the drug-resistant HCV is resistant to an anti-HCV agent. In certain embodiments, the anti-HCV agent is an interferon. In certain embodiments, the anti-HCV agent is ribaririn. In certain embodiments, the anti-HCV agent is amantadine. In certain embodiments, the anti-HCV agent is an interleukin. In certain embodiments, the anti-HCV agent is a phenanthrenequinone. In certain embodiments, the anti-HCV agent is a thiazolidine. In certain embodiments, the anti-HCV agent is a benzanilide. In certain embodiments, the anti-HCV agent is a helicase inhibitor. In certain embodiments, the anti-HCV agent is a nucleotide analogue. In certain embodiments, the anti-HCV agent is a gliotoxin. In certain embodiments, the anti-HCV agent is a cerulenin. In certain embodiments, the anti-HCV agent is an antisense phopshorothioate ologodexoynucleotide. In certain embodiments, the anti-HCV agent is an inhibitor of IRES-dependent translation. In certain embodiments, the anti-HCV agent is a ribozyme. In certain embodiments, the anti-HCV agent is a cyclophilin inhibitor. In certain embodiments, the anti-HCV agent is SYC-635.

In certain embodiments, the anti-HCV agent is a protease inhibitor. In certain embodiments, the anti-HCV agent is a cysteine protease inhibitor. In certain embodiments, the anti-HCV agent is a caspase inhibitor. In certain embodiments, the anti-HCV agent is GS 9450. In certain embodiments, the anti-HCV agent is a serine protease inhibitor. In certain embodiments, the anti-HCV agent is an NS3/4A serine protease inhibitor. In certain embodiments, the anti-HCV agent is a serine protease inhibitor selected from ABT-450, BI-201335, BMS-650032, boceprevir (SCH 503034), danoprevir (ITMN-191/R7227), GS-9256, IDX136, IDX316, IDX320, MK-5172, SCH900518, teleprevir (VX-950), TMC 435, vaniprevir (MK-7009), VX-985, and mixtures thereof.

In certain embodiments, the anti-HCV agent is a polymerase inhibitor. In certain embodiments, the anti-HCV agent is an NS5B polymerase inhibitor. In certain embodiments, the anti-HCV agent is a polymerase inhibitor selected from ABT-072, ABT-333, AG-02154, ANA598, ANA773, BI 207127, GS-9190, HCV-796, IDX184, IDX375, JTK-109, MK-0608, MK-3281, NM283, PF-868554, PSI-879, PSI-938, PSI-6130, PSI-7851, PSI-7977, R1626, R7128, RG7128, VCH-759, VCH-916, VX-222 (VCH-222), and mixtures thereof. In certain embodiments, the NS5B polymerase inhibitor is a nucleotide inhibitor. In certain embodiments, the NS5B polymerase inhibitor is a 2′C-methylnucleoside. In certain embodiments, the NS5B polymerase inhibitor is a non-nucleoside inhibitor. In certain embodiments, the NS5B polymerase inhibitor is a benzofuran, benzothiadiazine, or thiophene.

In certain embodiments, the anti-HCV agent is an NS5A inhibitor. In certain embodiments, the anti-HCV agent is an NS5A inhibitor selected from BMS-790052, BMS-824393, and mixtures thereof.

In certain embodiments, the drug-resistance of the HCV infection is caused by an HCV variant. In certain embodiments, the HCV variant contains an NS3 protein variant. In certain embodiments, the NS3 protein variant contains a mutation or deletion. In certain embodiments, the NS3 protein variant contains one or more mutations and/or deletions at the amino acid positions of 9, 16, 18, 23, 36, 39, 40, 41, 43, 54, 55, 65, 67, 70, 71, 80, 89, 109, 138, 155, 156, 162, 168, 170, 174, 176, 179, 260, and 489. In certain embodiments, the NS3 protein variant contains one or more mutations and/or deletions at the amino acid positions of 16, 23, 36, 39, 41, 43, 54, 55, 80, 89, 109, 138, 155, 156, 168, 170, 174, 176, 260, and 489. In certain embodiments, the NS3 protein variant contains one or more mutations and/or deletions at the amino acid positions of 36, 54, 155, 156, 168, and 170. In certain embodiments, the NS3 protein variant contains one, two, or more mutations and/or deletions, each independently selected from C16S, V23A, V36A, V36G, V36L, V36M, A39V, Q41R, F43C, F43I, F43S, F43V, T54A, T54S, V55A, Q80K, Q80G, Q80H, Q80L, Q80R, P89R, R109K, S138T, R155G, R155I, R155K, R155L, R155M, R155Q, R155S, R155T, A156G, A156I, A156S, A156T, A156V, D168A, D168E, D168G, D168H, D168I, D168N, D168T, D168V, D168Y, V170A, V170T, S174K, S174N, E176K, T260A, and S489L, provided that there is only one mutation or deletion at a given amino acid position in the NS3 protein variant. In certain embodiments, the NS3 protein variant contains one, two, or more mutations and/or deletions, each independently selected from R155K, A156S, A156T, D168V, and T260A, provided that there is only one mutation or deletion at a given amino acid position in the NS3 protein variant.

In certain embodiments, the HCV variant contains an NS4A protein variant. In certain embodiments, the NS4A protein variant contains a mutation or deletion. In certain embodiments, the NS4A protein variant contains a mutation at the amino acid position of 23. In certain embodiments, the NS4A protein variant contains the V23A mutation.

In certain embodiments, the HCV variant contains an NS4B protein variant. In certain embodiments, the NS4B protein variant contains a mutation or deletion. In certain embodiments, the NS4B protein variant contains a mutation at the amino acid position of 15. In certain embodiments, the NS4B protein variant contains the E15G mutation.

In certain embodiments, the HCV variant contains an NS5A protein variant. In certain embodiments, the NS5A protein variant contains a mutation or deletion. In certain embodiments, the NS5A protein variant contains one or more mutations and/or deletions at the amino acid positions of 23, 28, 30, 31, 32, 37, 54, 58, 63, and 93. In certain embodiments, the NS5A protein variant contains one or more mutations and/or deletions at the amino acid positions of 23, 24, 28, 30, 31, 32, 37, 54, 58, 63, 93, 295, 318, 320, 356, 404, and 442. In certain embodiments, the NS5A protein variant contains one or more mutations and/or deletions at the amino acid positions of 24, 28, 30, 31, 32, 54, 93, 295, and 318. In certain embodiments, the NS5A protein variant contains one, two, or more mutations and/or deletions, each independently selected from L23F, L28M, L28T, M28T, ΔQ30, Q30E, Q30H, Q30K, Q30R, AR30, R30E, R30Q, L31F, L31M, L31V, P32L, F37L, H54Y, Q54H, P58H, P58S, I63V, Y93C, Y93H, Y93N, and Y93S, provided that there is only one mutation or deletion at a given amino acid position in the NS5A protein variant. In certain embodiments, the NS5A protein variant contains one, two, or more mutations and/or deletions, each independently selected from L23F, K24E, L28M, L28T, M28T, ΔQ30, Q30E, Q30H, Q30K, Q30R, ΔR30, R30E, R30Q, L31F, L31M, L31V, P32L, F37L, H54Y, Q54H, P58H, P58S, 163V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from L23F, K24E, L28M, L28T, ΔQ30, Q30E, Q30H, Q30K, Q30R, L31F, L31M, L31V, P32L, F37L, H54Y, Q54H, P58H, P58S, I63V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from L23F, K24E, M28T, ΔR30, R30E, R30Q, L31F, L31M, L31V, P32L, F37L, H54Y, Q54H, P58H, P58S, I63V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from K24E, M28T, Q30E, Q30H, Q30K, Q30R, L31F, L31M, L31V, P32L, Y93C, Y93H, Y93N, E295G, and R318W, provided that there is only one mutation or deletion at a given amino acid position in the NS5A protein variant.

In certain embodiments, the subject being treated with a method provided herein is infected with a drug resistant genotype 2 HCV. In certain embodiments, the drug resistant genotype 2 HCV contains a NS5A protein mutation. In certain embodiments, the drug resistant genotype 2 HCV contains the L31M mutation in the NS5A protein.

In certain embodiments, the HCV variant contains an NS5B protein variant. In certain embodiments, the NS5B protein variant contains a mutation or deletion. In certain embodiments, the NS5B protein variant contains one or more mutations and/or deletions at the amino acid positions of 15, 95, 96, 142, 152, 156, 222, 223, 244, 282, 309, 310, 316, 320, 321, 326, 329, 333, 365, 411, 414, 415, 423, 445, 448, 451, 452, 495, 554, 558, and 559. In certain embodiments, the NS5B protein variant contains one or more mutations and/or deletions at the amino acid positions of 316, 414, and 423. In certain embodiments, the NS5B protein variant contains one, two, or more mutations and/or deletions, each independently selected from S15G, H95Q, H95R, S96T, N142T, G152E, P156L, R222Q, C223H, C223Y, D244N, S282T, Q309R, D310N, C316N, C316S, C316Y, L320I, V321I, S326G, T329I, A333E, S365A, S365T, N411S, M414I, M414L, M414T, F415Y, M423I, M423T, M423V, C445F, Y448H, C451R, Y452H, P495A, P495I, G554D, G554S, G558R, D559G, D559N, and D559S, provided that there is only one mutation or deletion at a given amino acid position in the NS5B protein variant. In certain embodiments, the NS5B protein variant contains one, two, or more mutations and/or deletions, each independently selected from C316Y, M414T, and M423T, provided that there is only one mutation or deletion at a given amino acid position in the NS5B protein variant.

In one embodiment, provided herein is a method for treating or preventing a hepatitis C virus infection in a subject, comprising administering to the subject the Compound or an isotopic variant thereof; or a pharmaceutically acceptable salt or solvate thereof; in an amount that is sufficient to provide a plasma concentration of the compound at steady state in the range from about 1 nM to about 1 μM, from about 2 nM to about 500 nM, from about 2 nM to about 200 nM, from about 2 nM to about 100 nM, or from about 2 nM to about 50 nM. In one embodiment, the amount of the Compound administered is sufficient to provide a plasma concentration at steady state in the range from about 1 nM to about 1 μM. In another embodiment, the amount of the Compound administered is sufficient to provide a plasma concentration at steady state in the range from about 2 nM to about 500 nM. In yet another embodiment, the amount of the Compound administered is sufficient to provide a plasma concentration at steady state in the range from about 2 nM to about 200 nM. In yet another embodiment, the amount of the Compound administered is sufficient to provide a plasma concentration at steady state in the range from about 2 nM to about 100 nM. In still another embodiment, the amount of the Compound administered is sufficient to provide a plasma concentration at steady state in the range from about 2 nM to about 50 nM. As used herein, the term “plasma concentration at steady state” is the concentration reached after a period of administration of a compound. Once steady state is reached, there are minor peaks and troughs on the time dependent curve of the plasma concentration of the compound.

In another embodiment, provided herein is a method for treating or preventing a hepatitis C virus infection in a subject, comprising administering to the subject the Compound or an isotopic variant thereof; or a pharmaceutically acceptable salt or solvate thereof; in an amount that is sufficient to provide a peak plasma concentration (a maximum plasma concentration) of the compound ranging from about 5 nM to about 1 μM, from about 5 nM to about 500 nM, from about 10 nM to about 200 nM, about 10 nM to about 100 nM, or from about 50 nM to about 100 nM. In one embodiment, the amount of the Compound administered is sufficient to provide a peak plasma concentration of the Compound ranging from about 5 nM to about 1 μM. In another embodiment, the amount of the Compound administered is sufficient to provide a peak plasma concentration of the Compound ranging from about 5 nM to about 500 nM. In yet another embodiment, the amount of the Compound administered is sufficient to provide a peak plasma concentration of the Compound ranging from about 10 nM to about 200 nM. In yet another embodiment, the amount of the Compound administered is sufficient to provide a peak plasma concentration of the Compound ranging from about 10 nM to about 100 nM. In still another embodiment, the amount of the Compound administered is sufficient to provide a peak plasma concentration of the Compound ranging from about 50 nM to about 100 nM.

In yet another embodiment, provided herein is a method for treating or preventing a hepatitis C virus infection in a subject, comprising administering to the subject the Compound or an isotopic variant thereof; or a pharmaceutically acceptable salt or solvate thereof; in an amount that is sufficient to provide a trough plasma concentration (a minimum plasma concentration) of the compound ranging from about 1 nM to about 500 nM, from about 2 nM to about 200 nM, from about 5 nM to about 100 nM, from about 1 nM to about 50 nM, from about 10 nM to about 50 nM, from about 1 nM to about 20 nM, or from about 1 nM to about 10 nM. In one embodiment, the amount of the Compound administered is sufficient to provide a trough plasma concentration of the compound ranging from about 1 nM to about 500 nM. In another embodiment, the amount of the Compound administered is sufficient to provide a trough plasma concentration of the compound ranging from about 2 nM to about 200 nM. In yet another embodiment, the amount of the Compound administered is sufficient to provide a trough plasma concentration of the compound ranging from about 5 nM to about 100 nM. In yet another embodiment, the amount of the Compound administered is sufficient to provide a trough plasma concentration of the compound ranging from about 1 nM to about 50 nM. In yet another embodiment, the amount of the Compound administered is sufficient to provide a trough plasma concentration of the compound ranging from about 10 nM to about 50 nM. In yet another embodiment, the amount of the Compound administered is sufficient to provide a trough plasma concentration of the compound ranging from about 1 nM to about 20 nM. In still another embodiment, the amount of the Compound administered is sufficient to provide a trough plasma concentration of the compound ranging from about 1 nM to about 10 nM.

In yet another embodiment, provided herein is a method for treating or preventing a hepatitis C virus infection in a subject, comprising administering to the subject the Compound or an isotopic variant thereof; or a pharmaceutically acceptable salt or solvate thereof; in an amount that is sufficient to provide an area under the curve (AUC) of the compound in the range from about 100 to about 10,000 ng·hr/mL, from about 100 to 5,000 ng·hr/mL, from about 100 to 2,000 ng·hr/mL, from about 200 to 2,000 ng·hr/mL, or from about 500 to 2,000 ng·hr/mL. In one embodiment, the amount of the Compound administered is sufficient to provide an AUC of the compound in the range from about 100 to about 10,000 ng·hr/mL. In another embodiment, the amount of the Compound administered is sufficient to provide an AUC of the compound in the range from about 100 to 5,000 ng·hr/mL. In yet another embodiment, the amount of the Compound administered is sufficient to provide an AUC of the compound in the range from about 100 to 2,000 ng·hr/mL. In yet another embodiment, the amount of the Compound administered is sufficient to provide an AUC of the compound in the range from about 200 to 2,000 ng·hr/mL. In still another embodiment, the amount of the Compound administered is sufficient to provide an AUC of the compound in the range from about 200 to 2,000 ng·hr/mL.

In yet another embodiment, provided herein is a method for treating, preventing, or ameliorating one or more symptoms of a liver disease or disorder associated with an HCV infection in a subject, comprising administering to the subject the Compound or an isotopic variant thereof; or a pharmaceutically acceptable salt or solvate thereof; in an amount that is sufficient to provide a plasma concentration of the compound at steady state in the range from about 1 nM to about 1 μM, from about 2 nM to about 500 nM, from about 5 nM to about 200 nM, from about 10 nM to about 100 nM, or from about 10 nM to about 50 nM. In one embodiment, the amount of the Compound administered is sufficient to provide a plasma concentration at steady state in the range from about 1 nM to about 1 μM. In another embodiment, the amount of the Compound administered is sufficient to provide a plasma concentration at steady state in the range from about 2 nM to about 500 nM. In yet another embodiment, the amount of the Compound administered is sufficient to provide a plasma concentration at steady state in the range from about 5 nM to about 200 nM. In yet another embodiment, the amount of the Compound administered is sufficient to provide a plasma concentration at steady state in the range from about 10 nM to about 100 nM. In still another embodiment, the amount of the Compound administered is sufficient to provide a plasma concentration at steady state in the range from about 10 nM to about 50 nM.

In yet another embodiment, provided herein is a method for treating, preventing, or ameliorating one or more symptoms of a liver disease or disorder associated with an HCV infection in a subject, comprising administering to the subject the Compound or an isotopic variant thereof; or a pharmaceutically acceptable salt or solvate thereof; in an amount that is sufficient to provide a peak plasma concentration of the compound ranging from about 5 nM to about 1 μM, from about 10 nM to about 500 nM, from about 20 nM to about 200 nM, or from about 50 nM to about 100 nM. In one embodiment, the amount of the Compound administered is sufficient to provide a peak plasma concentration of the Compound ranging from about 5 nM to about 1 μM. In another embodiment, the amount of the Compound administered is sufficient to provide a peak plasma concentration of the Compound ranging from about 10 nM to about 500 nM. In yet another embodiment, the amount of the Compound administered is sufficient to provide a peak plasma concentration of the Compound ranging from about 20 nM to about 200 nM. In still another embodiment, the amount of the Compound administered is sufficient to provide a peak plasma concentration of the Compound ranging from about 50 nM to about 100 nM.

In yet another embodiment, provided herein is a method for treating, preventing, or ameliorating one or more symptoms of a liver disease or disorder associated with an HCV infection in a subject, comprising administering to the subject the Compound or an isotopic variant thereof; or a pharmaceutically acceptable salt or solvate thereof; in an amount that is sufficient to provide a trough plasma concentration of the compound ranging from about 1 nM to about 500 nM, from about 2 nM to about 200 nM, from about 5 nM to about 100 nM, from about 10 nM to about 50 nM. In one embodiment, the amount of the Compound administered is sufficient to provide a trough plasma concentration of the compound ranging from about 1 nM to about 500 nM. In another embodiment, the amount of the Compound administered is sufficient to provide a trough plasma concentration of the compound ranging from about 2 nM to about 200 nM. In yet another embodiment, the amount of the Compound administered is sufficient to provide a trough plasma concentration of the compound ranging from about 5 nM to about 100 nM. In still another embodiment, the amount of the Compound administered is sufficient to provide a trough plasma concentration of the compound ranging from about 10 nM to about 50 nM.

In still another embodiment, provided herein is a method for treating, preventing, or ameliorating one or more symptoms of a liver disease or disorder associated with an HCV infection in a subject, comprising administering to the subject the Compound or an isotopic variant thereof; or a pharmaceutically acceptable salt or solvate thereof; in an amount that is sufficient to provide an area under the curve (AUC) of the compound in the range from about 100 to about 10,000 ng·hr/mL, from about 100 to 5,000 ng·hr/mL, from about 100 to 2,000 ng·hr/mL, from about 200 to 2,000 ng·hr/mL. In one embodiment, the amount of the Compound administered is sufficient to provide an AUC of the compound in the range from about 100 to about 10,000 ng·hr/mL. In another embodiment, the amount of the Compound administered is sufficient to provide an AUC of the compound in the range from about 100 to 5,000 ng·hr/mL. In yet another embodiment, the amount of the Compound administered is sufficient to provide an AUC of the compound in the range from about 100 to 2,000 ng·hr/mL. In still another embodiment, the amount of the Compound administered is sufficient to provide an AUC of the compound in the range from about 200 to 2,000 ng·hr/mL.

In certain embodiments, the HCV is, as described herein, drug resistant.

In one embodiment, provided herein is a method for inhibiting replication of a virus in a subject, comprising administering to the subject a therapeutically effective amount of the Compound or an isotopic variant thereof; or a pharmaceutically acceptable salt or solvate thereof; wherein the therapeutically effective amount is as defined herein.

In another embodiment, provided herein is a method for inhibiting replication of a virus in a subject, comprising administering to the subject a therapeutically effective amount of the Compound or an isotopic variant thereof; or a pharmaceutically acceptable salt or solvate thereof; wherein the therapeutically effective amount is as defined herein.

In yet another embodiment, provided herein is a method for inhibiting replication of a virus in a subject, comprising administering to the subject the Compound or an isotopic variant thereof; or a pharmaceutically acceptable salt or solvate thereof; in an amount that is sufficient to provide a plasma concentration of the compound at steady state in the range from about 1 nM to about 1 μM, from about 2 nM to about 500 nM, from about 5 nM to about 200 nM, from about 10 nM to about 100 nM, or from about 10 nM to about 50 nM. In one embodiment, the amount of the Compound administered is sufficient to provide a plasma concentration at steady state in the range from about 1 nM to about 1 μM. In another embodiment, the amount of the Compound administered is sufficient to provide a plasma concentration at steady state in the range from about 2 nM to about 500 nM. In yet another embodiment, the amount of the Compound administered is sufficient to provide a plasma concentration at steady state in the range from about 5 nM to about 200 nM. In yet another embodiment, the amount of the Compound administered is sufficient to provide a plasma concentration at steady state in the range from about 10 nM to about 100 nM. In still another embodiment, the amount of the Compound administered is sufficient to provide a plasma concentration at steady state in the range from about 10 nM to about 50 nM.

In yet another embodiment, provided herein is a method for inhibiting replication of a virus in a subject, comprising administering to the subject the Compound or an isotopic variant thereof; or a pharmaceutically acceptable salt or solvate thereof; in an amount that is sufficient to provide a peak plasma concentration of the compound ranging from about 5 nM to about 1 μM, from about 10 nM to about 500 nM, from about 20 nM to about 200 nM, or from about 50 nM to about 100 nM. In one embodiment, the amount of the Compound administered is sufficient to provide a peak plasma concentration of the Compound ranging from about 5 nM to about 1 μM. In another embodiment, the amount of the Compound administered is sufficient to provide a peak plasma concentration of the Compound ranging from about 10 nM to about 500 nM. In yet another embodiment, the amount of the Compound administered is sufficient to provide a peak plasma concentration of the Compound ranging from about 20 nM to about 200 nM. In still another embodiment, the amount of the Compound administered is sufficient to provide a peak plasma concentration of the Compound ranging from about 50 nM to about 100 nM.

In yet another embodiment, provided herein is a method for inhibiting replication of a virus in a subject, comprising administering to the subject the Compound or an isotopic variant thereof; or a pharmaceutically acceptable salt or solvate thereof; in an amount that is sufficient to provide a trough plasma concentration of the compound ranging from about 1 nM to about 500 nM, from about 2 nM to about 200 nM, from about 5 nM to about 100 nM, from about 10 nM to about 50 nM. In one embodiment, the amount of the Compound administered is sufficient to provide a trough plasma concentration of the compound ranging from about 1 nM to about 500 nM. In another embodiment, the amount of the Compound administered is sufficient to provide a trough plasma concentration of the compound ranging from about 2 nM to about 200 nM. In yet another embodiment, the amount of the Compound administered is sufficient to provide a trough plasma concentration of the compound ranging from about 5 nM to about 100 nM. In still another embodiment, the amount of the Compound administered is sufficient to provide a trough plasma concentration of the compound ranging from about 10 nM to about 50 nM.

In still another embodiment, provided herein is a method for inhibiting replication of a virus in a subject, comprising administering to the subject the Compound or an isotopic variant thereof; or a pharmaceutically acceptable salt or solvate thereof; in an amount that is sufficient to provide an area under the curve (AUC) of the compound in the range from about 100 to about 10,000 ng·hr/mL, from about 100 to 5,000 ng·hr/mL, from about 100 to 2,000 ng·hr/mL, from about 200 to 2,000 ng·hr/mL. In one embodiment, the amount of the Compound administered is sufficient to provide an AUC of the compound in the range from about 100 to about 10,000 ng·hr/mL. In another embodiment, the amount of the Compound administered is sufficient to provide an AUC of the compound in the range from about 100 to 5,000 ng·hr/mL. In yet another embodiment, the amount of the Compound administered is sufficient to provide an AUC of the compound in the range from about 100 to 2,000 ng·hr/mL. In still another embodiment, the amount of the Compound administered is sufficient to provide an AUC of the compound in the range from about 200 to 2,000 ng·hr/mL.

In certain embodiments, the virus is a hepatitis C virus. In certain embodiments, the virus is a drug resistant virus. In certain embodiments, the virus is a drug resistant hepatitis C virus.

In one embodiment, the hepatitis C virus is HCV genotype 1. In certain embodiments, the hepatitis C virus is HCV subtype 1a. In certain embodiments, the hepatitis C virus is HCV subtype 1b. In certain embodiments, the hepatitis C virus is HCV subtype 1c.

In another embodiment, the hepatitis C virus is HCV genotype 2. In certain embodiments, the hepatitis C virus is HCV subtype 2a. In certain embodiments, the hepatitis C virus is HCV subtype 2b. In certain embodiments, the hepatitis C virus is HCV subtype 2c.

In yet another embodiment, the hepatitis C virus is HCV genotype 3. In certain embodiments, the hepatitis C virus is HCV subtype 3a. In certain embodiments, the hepatitis C virus is HCV subtype 3b.

In yet another embodiment, the hepatitis C virus is HCV genotype 4. In certain embodiments, the hepatitis C virus is HCV subtype 4a. In certain embodiments, the hepatitis C virus is HCV subtype 4b. In certain embodiments, the hepatitis C virus is HCV subtype 4c. In certain embodiments, the hepatitis C virus is HCV subtype 4d. In certain embodiments, the hepatitis C virus is HCV subtype 4e.

In yet another embodiment, the hepatitis C virus is HCV genotype 5. In yet another embodiment, the hepatitis C virus is HCV subtype 5a.

In yet another embodiment, the hepatitis C virus is HCV genotype 6. In yet another embodiment, the hepatitis C virus is HCV subtype 6a.

In yet another embodiment, the hepatitis C virus is HCV genotype 7. In yet another embodiment, the hepatitis C virus is HCV subtype 7a.

In yet another embodiment, the hepatitis C virus is HCV genotype 8. In yet another embodiment, the hepatitis C virus is HCV subtype 8a. In yet another embodiment, the hepatitis C virus is HCV subtype 8b.

In yet another embodiment, the hepatitis C virus is HCV genotype 9. In yet another embodiment, the hepatitis C virus is HCV subtype 9a.

In yet another embodiment, the hepatitis C virus is HCV genotype 10. In yet another embodiment, the hepatitis C virus is HCV subtype 10a.

In still another embodiment, the hepatitis C virus is HCV genotype 11. In yet another embodiment, the hepatitis C virus is HCV subtype 11a.

In one embodiment, the HCV is a HCV variant. In another embodiment, the virus is a HCV variant.

In one embodiment, the HCV variant is a variant of HCV genotype 1. In certain embodiments, the HCV variant is a variant of HCV subtype 1a. In certain embodiments, the HCV variant is a variant of HCV subtype 1b. In certain embodiments, the HCV variant is a variant of HCV subtype 1c.

In certain embodiments, the HCV variant is a variant of HCV subtype 1a, which contains an NS5A protein variant. In certain embodiments, the NS5A protein variant contains a mutation or deletion. In certain embodiments, the NS5A protein variant contains one or more mutations and/or deletions at the amino acid positions of 28, 30, 31, 32, 54, and 93. In certain embodiments, the NS5A protein variant contains one or more mutations and/or deletions at the amino acid positions of 23, 24, 28, 30, 31, 32, 37, 54, 58, 63, 93, 295, 318, 320, 356, 404, and 442. In certain embodiments, the NS5A protein variant contains one or more mutations and/or deletions at the amino acid positions of 24, 28, 30, 31, 32, 54, 93, 295, and 318. In certain embodiments, the NS5A protein variant contains one, two, or more mutations and/or deletions, each independently selected from M28T, ΔQ30, Q30E, Q30H, Q30K, Q30R, L31F, L31M, L31V, P32L, H54Y, Y93C, Y93H, and Y93N, provided that there is only one mutation or deletion at a given amino acid position in the NS5A protein variant. In certain embodiments, the NS5A protein variant contains one, two, or more mutations and/or deletions, each independently selected from L23F, K24E, L28M, L28T, M28T, ΔQ30, Q30E, Q30H, Q30K, Q30R, ΔR30, R30E, R30Q, L31F, L31M, L31V, P32L, F37L, H54Y, Q54H, P58H, P58S, I63V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from L23F, K24E, L28M, L28T, ΔQ30, Q30E, Q30H, Q30K, Q30R, L31F, L31M, L31V, P32L, F37L, H54Y, Q54H, P58H, P58S, I63V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from L23F, K24E, M28T, ΔR30, R30E, R30Q, L31F, L31M, L31V, P32L, F37L, H54Y, Q54H, P58H, P58S, I63V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from K24E, M28T, Q30E, Q30H, Q30K, Q30R, L31F, L31M, L31V, P32L, Y93C, Y93H, Y93N, E295G, and R318W, provided that there is only one mutation or deletion at a given amino acid position in the NS5A protein variant. In certain embodiments, the NS5A protein variant contains one or more mutations at the amino acid positions of 28, 30, 31, 32, and 93. In certain embodiments, the NS5A protein variant contains one, two, or more mutations, each independently selected from M28T, Q30E, Q30H, Q30K, Q30R, L31F, L31M, L31V, P32L, Y93C, Y93H, and Y93N, provided that there is only one mutation at a given amino acid position in the NS5A protein variant. In certain embodiments, the NS5A protein variant contains one or more mutations at the amino acid positions of 24, 28, 30, 31, 32, 93, 295, and 318. In certain embodiments, the NS5A protein variant contains one, two, or more mutations, each independently selected from K24E, M28T, Q30E, Q30H, Q30K, Q30R, L31F, L31M, L31V, P32L, Y93C, Y93H, Y93N, E295G, and R318W, provided that there is only one mutation at a given amino acid position in the NS5A protein variant.

In certain embodiments, the HCV variant is a variant of HCV subtype 1b, which contains an NS5A protein variant. In certain embodiments, the NS5A protein variant contains a mutation or deletion. In certain embodiments, the NSSA protein variant contains one or more mutations and/or deletions at the amino acid positions of 23, 28, 30, 31, 32, 37, 54, 58, 63, and 93. In certain embodiments, the NS5A protein variant contains one or more mutations and/or deletions at the amino acid positions of 23, 24, 28, 30, 31, 32, 37, 54, 58, 63, 93, 295, 318, 320, 356, 404, and 442. In certain embodiments, the NS5A protein variant contains one or more mutations and/or deletions at the amino acid positions of 24, 28, 30, 31, 32, 54, 93, 295, and 318. In certain embodiments, the NS5A protein variant contains one, two, or more mutations and/or deletions, each independently selected from L23F, L28M, L28T, ΔR30, R30E, R30Q, L31F, L31M, L31V, P32L, F37L, Q54H, P58H, P58S, I63V, Y93C, Y93H, Y93N, and Y93S, provided that there is only one mutation or deletion at a given amino acid position in the NS5A protein variant. In certain embodiments, the NS5A protein variant contains one, two, or more mutations and/or deletions, each independently selected from L23F, K24E, L28M, L28T, M28T, ΔQ30, Q30E, Q30H, Q30K, Q30R, ΔR30, R30E, R30Q, L31F, L31M, L31V, P32L, F37L, H54Y, Q54H, P58H, P58S, I63V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from L23F, K24E, L28M, L28T, ΔQ30, Q30E, Q30H, Q30K, Q30R, L31F, L31M, L31V, P32L, F37L, H54Y, Q54H, P58H, P58S, I63V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from L23F, K24E, M28T, ΔR30, R30E, R30Q, L31F, L31M, L31V, P32L, F37L, H54Y, Q54H, P58H, P58S, I63V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from K24E, M28T, Q30E, Q30H, Q30K, Q30R, L31F, L31M, L31V, P32L, Y93C, Y93H, Y93N, E295G, and R318W, provided that there is only one mutation or deletion at a given amino acid position in the NS5A protein variant. In certain embodiments, the NS5A protein variant contains one or more mutations at the amino acid positions of 28, 30, 31, 32, and 93. In certain embodiments, the NS5A protein variant contains one, two, or more mutations, each independently selected from L28T, R30E, L31F, L31M, L31V, P32L, Y93C, Y93H, and Y93N, provided that there is only one mutation at a given amino acid position in the NS5A protein variant. In certain embodiments, the NS5A protein variant contains one or more mutations at the amino acid positions of 24, 28, 30, 31, 32, 93, 295, and 318. In certain embodiments, the NS5A protein variant contains one, two, or more mutations, each independently selected from K24E, M28T, Q30E, Q30H, Q30K, Q30R, L31F, L31M, L31V, P32L, Y93C, Y93H, Y93N, E295G, and R318W, provided that there is only one mutation at a given amino acid position in the NS5A protein variant.

In another embodiment, the HCV variant is a variant of HCV genotype 2. In certain embodiments, the HCV variant is a variant of HCV subtype 2a. In certain embodiments, the HCV variant is a variant of HCV subtype 2b. In certain embodiments, the HCV variant is a variant of HCV subtype 2c.

In yet another embodiment, the HCV variant is a variant of HCV genotype 3. In certain embodiments, the HCV variant is a variant of HCV subtype 3a. In certain embodiments, the HCV variant is a variant of HCV subtype 3b.

In yet another embodiment, the HCV variant is a variant of HCV genotype 4. In certain embodiments, the HCV variant is a variant of HCV subtype 4a. In certain embodiments, the HCV variant is a variant of HCV subtype 4b. In certain embodiments, the HCV variant is a variant of HCV subtype 4c. In certain embodiments, the HCV variant is a variant of HCV subtype 4d. In certain embodiments, the HCV variant is a variant of HCV subtype 4e.

In yet another embodiment, the HCV variant is a variant of HCV genotype 5. In yet another embodiment, the HCV variant is a variant of HCV subtype 5a.

In yet another embodiment, the HCV variant is a variant of HCV genotype 6. In yet another embodiment, the HCV variant is a variant of HCV subtype 6a.

In yet another embodiment, the HCV variant is a variant of HCV genotype 7. In yet another embodiment, the HCV variant is a variant of HCV subtype 7a.

In yet another embodiment, the HCV variant is a variant of HCV genotype 8. In yet another embodiment, the HCV variant is a variant of HCV subtype 8a. In yet another embodiment, the HCV variant is a variant of HCV subtype 8b.

In yet another embodiment, the HCV variant is a variant of HCV genotype 9. In yet another embodiment, the HCV variant is a variant of HCV subtype 9a.

In yet another embodiment, the HCV variant is a variant of HCV genotype 10. In yet another embodiment, the HCV variant is a variant of HCV subtype 10a.

In still another embodiment, the HCV variant is a variant of HCV genotype 11. In yet another embodiment, the HCV variant is a variant of HCV subtype 11a.

In certain embodiments, administration of a therapeutically effective amount of the Compound or an isotopic variant thereof; or a pharmaceutically acceptable salt or solvate thereof, results in a 90%, 99%, or 99.9% reduction in the replication of the virus relative to a subject without administration of the compound, as determined at 1 day, 2 days, 3 days, 4 days, 5 days, 10 days, 15 days, or 30 days after the administration by a method known in the art, e.g., determination of viral titer. In certain embodiments, the administration of the Compound or an isotopic variant thereof; or a pharmaceutically acceptable salt or solvate thereof, results in a 90% reduction in the replication of the virus relative to a subject without administration of the compound, as determined at 1 day after the administration. In certain embodiments, the administration of the Compound or an isotopic variant thereof; or a pharmaceutically acceptable salt or solvate thereof, results in a 99% reduction in the replication of the virus relative to a subject without administration of the compound, as determined at 1 day after the administration. In certain embodiments, the administration of the Compound or an isotopic variant thereof; or a pharmaceutically acceptable salt or solvate thereof, results in a 99.9% reduction in the replication of the virus relative to a subject without administration of the compound, as determined at 1 day after the administration.

In certain embodiments, administration of a therapeutically effective amount of the Compound or an isotopic variant thereof; or a pharmaceutically acceptable salt or solvate thereof, results in a 10-fold (1 log₁₀), 100-fold (2 log₁₀), 1,000-fold (3 log₁₀), or 10,000-fold (4 log₁₀) reduction in the replication of the virus relative to a subject without administration of the compound, as determined at 1 day, 2 days, 3 days, 4 days, 5 days, 10 days, 15 days, or 30 days after the administration by a method known in the art. In certain embodiments, the administration of the Compound or an isotopic variant thereof; or a pharmaceutically acceptable salt or solvate thereof, results in a 1 log₁₀ reduction in the replication of the virus relative to a subject without administration of the compound, as determined at 1 day after the administration. In certain embodiments, the administration of the Compound or an isotopic variant thereof; or a pharmaceutically acceptable salt or solvate thereof, results in a 2 log₁₀ reduction in the replication of the virus relative to a subject without administration of the compound, as determined at 1 day after the administration. In certain embodiments, the administration of the Compound or an isotopic variant thereof; or a pharmaceutically acceptable salt or solvate thereof, results in a 3 log₁₀ reduction in the replication of the virus relative to a subject without administration of the compound, as determined at 1 day after the administration. In certain embodiments, the administration of the Compound or an isotopic variant thereof; or a pharmaceutically acceptable salt or solvate thereof, results in a 4 log₁₀ reduction in the replication of the virus relative to a subject without administration of the compound, as determined at 1 day after the administration.

In certain embodiments, administration of a therapeutically effective amount of the Compound or an isotopic variant thereof; or a pharmaceutically acceptable salt or solvate thereof, results in a 90%, 99%, or 99.9% reduction in the viral titer relative to a subject without administration of the compound, as determined at 1 day, 2 days, 3 days, 4 days, 5 days, 10 days, 15 days, or 30 days after the administration by a method known in the art. In certain embodiments, the administration of the Compound or an isotopic variant thereof; or a pharmaceutically acceptable salt or solvate thereof, results in a 90% reduction in the viral titer relative to a subject without administration of the compound, as determined at 1 day after the administration. In certain embodiments, the administration of the Compound or an isotopic variant thereof; or a pharmaceutically acceptable salt or solvate thereof, results in a 99% reduction in the viral titer relative to a subject without administration of the compound, as determined at 1 day after the administration. In certain embodiments, the administration of the Compound or an isotopic variant thereof; or a pharmaceutically acceptable salt or solvate thereof, results in a 99.9% reduction in the viral titer relative to a subject without administration of the compound, as determined at 1 day after the administration.

In certain embodiments, administration of a therapeutically effective amount of the Compound or an isotopic variant thereof; or a pharmaceutically acceptable salt or solvate thereof, results in a 10-fold (1 log₁₀), 100-fold (2 log₁₀), 1,000-fold (3 log₁₀), or 10,000-fold (4 log₁₀) in the viral titer relative to a subject without administration of the compound, as determined at 1 day, 2 days, 3 days, 4 days, 5 days, 10 days, 15 days, or 30 days after the administration by a method known in the art. In certain embodiments, the administration of the Compound or an isotopic variant thereof; or a pharmaceutically acceptable salt or solvate thereof, results in a 1 log₁₀ reduction in the viral titer relative to a subject without administration of the compound, as determined at 1 day after the administration. In certain embodiments, the administration of the Compound or an isotopic variant thereof; or a pharmaceutically acceptable salt or solvate thereof, results in a 2 log₁₀ reduction in the viral titer relative to a subject without administration of the compound, as determined at 1 day after the administration. In certain embodiments, the administration of the Compound or an isotopic variant thereof; or a pharmaceutically acceptable salt or solvate thereof, results in a 3 log₁₀ reduction in the viral titer relative to a subject without administration of the compound, as determined at 1 day after the administration. In certain embodiments, the administration of the Compound or an isotopic variant thereof; or a pharmaceutically acceptable salt or solvate thereof, results in a 4 log₁₀ reduction in the viral titer relative to a subject without administration of the compound, as determined at 1 day after the administration.

In certain embodiments, the subject to be treated with one of the methods provided herein has not been treated with anti-HCV therapy (i.e., treatment-naive) prior to the administration of the Compound or an isotopic variant thereof, or a pharmaceutically acceptable salt or solvate thereof. In certain embodiments, the subject to be treated with one of the methods provided herein has been treated with anti-HCV therapy prior to the administration of the Compound or an isotopic variant thereof, or a pharmaceutically acceptable salt or solvate thereof. In certain embodiments, the subject to be treated with one of the methods provided herein has not been treated with an NS5A inhibitor prior to the administration of the Compound or an isotopic variant thereof, or a pharmaceutically acceptable salt or solvate thereof. In certain embodiments, the subject to be treated with one of the methods provided herein has been treated with an NS5A inhibitor prior to the administration of the Compound or an isotopic variant thereof, or a pharmaceutically acceptable salt or solvate thereof.

In certain embodiments, the subject is a human.

In certain embodiments, the subject has an IL28B (interleukin 28B) CC genotype. In certain embodiments, the subject has an IL28B CT genotype. In certain embodiments, the subject has an IL28B TT genotype.

The methods provided herein encompass treating a subject regardless of patient's age, although some diseases or disorders are more common in certain age groups.

Depending on the disease to be treated and the subject's condition, the Compound or an isotopic variant thereof, or a pharmaceutically acceptable salt or solvate thereof, may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. The Compound or an isotopic variant thereof, or a pharmaceutically acceptable salt or solvate thereof, may be formulated, alone or together, in suitable dosage unit with pharmaceutically acceptable excipients, carriers, adjuvants and vehicles, appropriate for each route of administration. In one embodiment, the Compound or an isotopic variant thereof, or a pharmaceutically acceptable salt or solvate thereof, is administered orally. In another embodiment, the Compound or an isotopic variant thereof, or a pharmaceutically acceptable salt or solvate thereof, is administered parenterally. In yet another embodiment, the Compound or an isotopic variant thereof, or a pharmaceutically acceptable salt or solvate thereof, is administered intravenously.

The Compound or an isotopic variant thereof, or a pharmaceutically acceptable salt or solvate thereof, can be delivered as a single dose, such as, e.g., a single bolus injection, or a single oral tablet or pill; or over time such as, e.g., continuous infusion over time or divided bolus doses over time. The compound can be administered repetitively if necessary, for example, until the patient experiences stable disease or regression, or until the patient experiences disease progression or unacceptable toxicity. Stable disease or lack thereof is determined by methods known in the art such as evaluation of patient's symptoms, physical examination, or measuring patient's viral level.

The Compound or an isotopic variant thereof, or a pharmaceutically acceptable salt or solvate thereof, can be administered once daily (QD), or divided into multiple daily doses, such as twice daily (BID), three times daily (TID), and four times daily (QID).

In certain embodiments, the Compound or an isotopic variant thereof, or a pharmaceutically acceptable salt or solvate thereof, is administered once daily (QD). In certain embodiments, the Compound or an isotopic variant thereof, or a pharmaceutically acceptable salt or solvate thereof, is administered twice a day (BID). In certain embodiments, the Compound or an isotopic variant thereof, or a pharmaceutically acceptable salt or solvate thereof, is administered three times a day (TID). In certain embodiments, the Compound or an isotopic variant thereof, or a pharmaceutically acceptable salt or solvate thereof, is administered four times a day (QID).

In certain embodiments, the Compound or an isotopic variant thereof, or a pharmaceutically acceptable salt or solvate thereof, is administered on an empty stomach. In certain embodiments, the Compound or an isotopic variant thereof, or a pharmaceutically acceptable salt or solvate thereof, is administered at least about one hour before eating or at least about two hours after eating. In certain embodiments, the Compound or an isotopic variant thereof, or a pharmaceutically acceptable salt or solvate thereof, is administered at least about one hour before eating. In certain embodiments, the Compound or an isotopic variant thereof, or a pharmaceutically acceptable salt or solvate thereof, is administered at least about two hours after eating.

### Combination Therapy

The compounds provided herein may also be combined or used in combination with other therapeutic agents useful in the treatment and/or prevention of an HCV infection.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a compound provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

As used herein, the term “synergistic” includes a combination of a compound provided herein and another therapy (e.g., a prophylactic or therapeutic agent) which has been or is currently being used to prevent, treat, or manage a condition, disorder, or disease, which is more effective than the additive effects of the therapies. A synergistic effect of a combination of therapies (e.g., a combination of prophylactic or therapeutic agents) permits the use of lower dosages of one or more of the therapies and/or less frequent administration of said therapies to a subject with a condition, disorder, or disease. The ability to utilize lower dosages of a therapy (e.g., a prophylactic or therapeutic agent) and/or to administer said therapy less frequently reduces the toxicity associated with the administration of said therapy to a subject without reducing the efficacy of said therapy in the prevention, treatment, or management of a condition, disorder, or disease). In addition, a synergistic effect can result in improved efficacy of agents in the prevention, treatment, or management of a condition, disorder, or disease. Finally, a synergistic effect of a combination of therapies (e.g., a combination of prophylactic or therapeutic agents) may avoid or reduce adverse or unwanted side effects associated with the use of either therapy alone.

The compound provided herein can be administered in combination or alternation with another therapeutic agent, such as an anti-HCV agent. In combination therapy, effective dosages of two or more agents are administered together, whereas in alternation or sequential-step therapy, an effective dosage of each agent is administered serially or sequentially. The dosages given will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

It has been recognized that drug-resistant variants of HCV can emerge after prolonged treatment with an antiviral agent. Drug resistance most typically occurs due to the mutation of a gene that encodes for an enzyme used in viral replication. The efficacy of a drug against the viral infection can be prolonged, augmented, or restored by administering the compound in combination or alternation with a second, and perhaps third, antiviral compound that induces a different mutation from that caused by the principle drug. Alternatively, the pharmacokinetics, biodistribution, or other parameters of the drug can be altered by such combination or alternation therapy. In general, combination therapy is typically preferred over alternation therapy because it induces multiple simultaneous stresses on the virus.

In certain embodiments, the pharmaceutical compositions provided herein further comprise a second antiviral agent as described herein. In certain embodiments, the compound provided herein is combined with one or more agents selected from the group consisting of an interferon, ribavirin, amantadine, an interleukin, an NS3 protease inhibitor, a cysteine protease inhibitor, a phenanthrenequinone, a thiazolidine, a benzanilide, a helicase inhibitor, a polymerase inhibitor, a nucleotide analogue, a gliotoxin, a cerulenin, an antisense phosphorothioate oligodeoxynucleotide, an inhibitor of IRES-dependent translation, and a ribozyme. In one embodiment, the second antiviral agent is an interferon. In another embodiment, the interferon is selected from the group consisting of pegylated interferon alpha 2a, interferon alfacon-1, natural interferon, ALBUFERON®, interferon beta-1a, omega interferon, interferon alpha, interferon gamma, interferon tau, interferon delta, and interferon gamma-1b.

In certain embodiments, the compound provided herein is combined with an HCV protease inhibitor, including, but not limited to, BI 201335 (Boehringer Ingelheim); TMC 435 or TMC 435350 (Medivir/Tibotec); ITMN 191/R7227 (InterMune); MK 7009 (Merck); SCH 5034/SCH 503034/Boceprevir and SCH 900518/narlaprevir (Schering); VX950/telaprevir (Vertex); substrate-based NS3 protease inhibitors as disclosed in DE 19914474, WO 98/17679, WO 98/22496, WO 99/07734, and Attwood et al., Antiviral Chemistry and Chemotherapy 1999, 10, 259-273; non-substrate-based NS3 protease inhibitors, including 2,4,6-trihydroxy-3-nitro-benzamide derivatives (Sudo et al., Biochem. Biophys. Res. Commun. 1997, 238, 643-647), a phenanthrenequinone (Chu et al., Tetrahedron Letters 1996, 37, 7229-7232), RD3-4082, RD3-4078, SCH 68631, and SCH 351633 (Chu et al., Bioorganic and Medicinal Chemistry Letters 1999, 9, 1949-1952); and Eglin C, a potent serine protease inhibitor (Qasim et al., Biochemistry 1997, 36, 1598-1607).

Other suitable protease inhibitors for the treatment of HCV include those disclosed in, for example, U.S. Pat. No. 6,004,933, which discloses a class of cysteine protease inhibitors of HCV endopeptidase 2.

Additional hepatitis C virus NS3 protease inhibitors include those disclosed in, for example, Llinàs-Brunet et al., Bioorg. Med. Chem. Lett. 1998, 8, 1713-1718; Steinkühler et al., Biochemistry 1998, 37, 8899-8905; U.S. Pat. Nos. 5,538,865; 5,990,276; 6,143,715; 6,265,380; 6,323,180; 6,329,379; 6,410,531; 6,420,380; 6,534,523; 6,608,027; 6,642,204; 6,653,295; 6,727,366; 6,838,475; 6,846,802; 6,867,185; 6,869,964; 6,872,805; 6,878,722; 6,908,901; 6,911,428; 6,995,174; 7,012,066; 7,041,698; 7,091,184; 7,169,760; 7,176,208; 7,208,600; and 7,491,794; U.S. Pat. App. Pub. Nos.: 2002/0016294, 2002/0016442; 2002/0032175; 2002/0037998; 2004/0229777; 2005/0090450; 2005/0153877; 2005/176648; 2006/0046956; 2007/0021330; 2007/0021351; 2007/0049536; 2007/0054842; 2007/0060510; 2007/0060565; 2007/0072809; 2007/0078081; 2007/0078122; 2007/0093414; 2007/0093430; 2007/0099825; 2007/0099929; 2007/0105781, 2008/0152622, 2009/0035271, 2009/0035272, 2009/0047244, 2009/0111969, 2009/0111982, 2009/0123425, 2009/0130059, 2009/0148407, 2009/0156800, 2009/0169510, 2009/0175822, 2009/0180981, and 2009/0202480; U.S. patent application Ser. No. 12/365,127; and International Pat. App. Pub. Nos.: WO 98/17679; WO 98/22496; WO 99/07734; WO 00/09543; WO 00/59929; WO 02/08187; WO 02/08251; WO 02/08256; WO 02/08198; WO 02/48116; WO 02/48157; WO 02/48172; WO 02/60926; WO 03/53349; WO 03/64416; WO 03/64455; WO 03/64456; WO 03/66103; WO 03/99274; WO 03/99316; WO 2004/032827; WO 2004/043339; WO 2005/037214; WO 2005/037860; WO 2006/000085; WO 2006/119061; WO 2006/122188; WO 2007/001406; WO 2007/014925; WO 2007/014926; WO 2007/015824, WO 2007/056120, WO 2008/019289, WO 2008/021960, WO 2008/022006, WO 2008/086161, WO 2009/053828, WO 2009/058856, WO 2009/073713, WO 2009/073780, WO 2009/080542, WO 2009/082701, WO 2009/082697, and WO 2009/085978; the disclosure of each of which is incorporated herein by reference in its entirety.

Other protease inhibitors include thiazolidine derivatives, such as RD-1-6250, RD4 6205, and RD4 6193, which show relevant inhibition in a reverse-phase HPLC assay with an NS3/4A fusion protein and NS5A/5B substrate (Sudo et al., Antiviral Research 1996, 32, 9-18); and thiazolidines and benzanilides identified in Kakiuchi et al., FEBS Lett. 1998, 421, 217-220; and Takeshita et al., Analytical Biochemistry 1997, 247, 242-246.

Suitable helicase inhibitors include, but are not limited to, those disclosed in U.S. Pat. No. 5,633,358; and International Pat. App. Pub. No. WO 97/36554.

Suitable nucleotide polymerase inhibitors include, but are not limited to, gliotoxin (Ferrari et al., Journal of Virology 1999, 73, 1649-1654) and cerulenin (Lohmann et al., Virology 1998, 249, 108-118).

Suitable interfering RNA (iRNA) based antivirals include, but are not limited to, short interfering RNA (siRNA) based antivirals, such as Sirna-034 and those described in International Pat. App. Pub. Nos. WO/03/070750 and WO 2005/012525, and U.S. Pat. App. Pub. No. 2004/0209831.

Suitable antisense phosphorothioate oligodeoxynucleotides (S-ODN) complementary to sequence stretches in the 5′ non-coding region (NCR) of HCV virus include, but are not limited to those described in Alt et al., Hepatology 1995, 22, 707-717, and nucleotides 326-348 comprising the 3′ end of the NCR and nucleotides 371-388 located in the core coding region of HCV RNA (Alt et al., Archives of Virology 1997, 142, 589-599; and Galderisi et al., Journal of Cellular Physiology 1999, 181, 251-257);

Suitable inhibitors of IRES-dependent translation include, but are not limited to, those described in Japanese Pat. App. Pub. Nos.: JP 08268890 and JP 10101591.

Suitable ribozymes include those disclosed in, for example, U.S. Pat. Nos. 6,043,077; 5,869,253; and 5,610,054.

Suitable nucleoside analogs include, but are not limited to, the compounds described in U.S. Pat. Nos. 6,660,721; 6,777,395; 6,784,166; 6,846,810; 6,927,291; 7,094,770; 7,105,499; 7,125,855; and 7,202,224; U.S. Pat. App. Pub. Nos. 2004/0121980; 2005/0009737; 2005/0038240; and 2006/0040890; and International Pat. App. Pub. Nos: WO 99/43691; WO 01/32153; WO 01/60315; WO 01/79246; WO 01/90121, WO 01/92282, WO 02/18404; WO 02/32920, WO 02/48165, WO 02/057425; WO 02/057287; WO 2004/002422, WO 2004/002999, and WO 2004/003000.

Other miscellaneous compounds that can be used as second agents include, for example, 1-amino-alkylcyclohexanes (U.S. Pat. No. 6,034,134), alkyl lipids (U.S. Pat. No. 5,922,757), vitamin E and other antioxidants (U.S. Pat. No. 5,922,757), squalene, amantadine, bile acids (U.S. Pat. No. 5,846,964), N-(phosphonacetyl)-L-aspartic acid (U.S. Pat. No. 5,830,905), benzenedicarboxamides (U.S. Pat. No. 5,633,388), polyadenylic acid derivatives (U.S. Pat. No. 5,496,546), 2′,3′-dideoxyinosine (U.S. Pat. No. 5,026,687), benzimidazoles (U.S. Pat. No. 5,891,874), plant extracts (U.S. Pat. Nos. 5,725,859; 5,837,257; and 6,056,961), and piperidines (U.S. Pat. No. 5,830,905).

In certain embodiments, one or more compounds provided herein are administered in combination or alternation with an anti-hepatitis C virus interferon, including, but not limited to, INTRON® A (interferon alfa-2b), PEGASYS® (Peginterferon alfa-2a) ROFERON® A (recombinant interferon alfa-2a), INFERGEN® (interferon alfacon-1), and PEG-INTRON® (pegylated interferon alfa-2b). In one embodiment, the anti-hepatitis C virus interferon is INFERGEN®, IL-29 (PEG-Interferon lambda), R7025 (Maxy-alpha), BELEROFON®, oral interferon alpha, BLX-883 (LOCTERON®), omega interferon, MULTIFERON®, medusa interferon, ALBUFERON®, or REBIF®.

In certain embodiments, one or more compounds provided herein are administered in combination or alternation with an anti-hepatitis C virus polymerase inhibitor, such as ribavirin, viramidine, NM 283 (valopicitabine), PSI-6130, R1626, HCV-796, R7128, and those as disclosed in U.S. Pat. App. Pub. Nos. 2009/0081158 and 2009/0238790, the disclosure of each of which is incorporated herein by reference in its entirety.

In certain embodiments, the one or more compounds provided herein are administered in combination with ribavirin and an anti-hepatitis C virus interferon, such as INTRON® A (interferon alfa-2b), PEGASYS® (Peginterferon alfa-2a), ROFERON® A (recombinant interferon alfa-2a), INFERGEN® (interferon alfacon-1), and PEG-INTRON® (pegylated interferon alfa-2b),

In certain embodiments, one or more compounds provided herein are administered in combination or alternation with an anti-hepatitis C virus protease inhibitor, such as ITMN-191, SCH 503034, VX950 (telaprevir), and TMC 435.

In certain embodiments, one or more compounds provided herein are administered in combination or alternation with an anti-hepatitis C virus vaccine, including, but not limited to, TG4040, PEVIPRO™, CGI-5005, HCV/MF59, GV1001, IC41, and INNOOl01 (E1).

In certain embodiments, one or more compounds provided herein are administered in combination or alternation with an anti-hepatitis C virus monoclonal antibody, such as AB68 and XTL-6865 (formerly HepX-C); or an anti-hepatitis C virus polyclonal antibody, such as cicavir.

In certain embodiments, one or more compounds provided herein are administered in combination or alternation with an anti-hepatitis C virus immunomodulator, such as ZADAXIN® (thymalfasin), NOV-205, and oglufanide.

In certain embodiments, one or more compounds provided herein are administered in combination or alternation with NEXAVAR®, doxorubicin, PI-88, amantadine, JBK-122, VGX-410C, MX-3253 (celgosivir), SUVUS® (BIVN-401 or virostat), PF-03491390 (formerly IDN-6556), G126270, UT-231B, DEBIO-025, EMZ702, ACH-0137171, MitoQ, ANA975, AVI-4065, bavituximab (tarvacin), ALINIA® (nitrazoxanide), and PYN17.

The compounds provided herein can also be administered in combination with other classes of compounds, including, but not limited to, (1) alpha-adrenergic agents; (2) antiarrhythmic agents; (3) anti-atherosclerotic agents, such as ACAT inhibitors; (4) antibiotics, such as anthracyclines, bleomycins, mitomycin, dactinomycin, and plicamycin; (5) anticancer agents and cytotoxic agents, e.g., alkylating agents, such as nitrogen mustards, alkyl sulfonates, nitrosoureas, ethylenimines, and triazenes; (6) anticoagulants, such as acenocoumarol, argatroban, bivalirudin, lepirudin, fondaparinux, heparin, phenindione, warfarin, and ximelagatran; (7) anti-diabetic agents, such as biguanides (e.g., metformin), glucosidase inhibitors (e.g., acarbose), insulins, meglitinides (e.g., repaglinide), sulfonylureas (e.g., glimepiride, glyburide, and glipizide), thiozolidinediones (e.g., troglitazone, rosiglitazone, and pioglitazone), and PPAR-gamma agonists; (8) antifungal agents, such as amorolfine, amphotericin B, anidulafungin, bifonazole, butenafine, butoconazole, caspofungin, ciclopirox, clotrimazole, econazole, fenticonazole, filipin, fluconazole, isoconazole, itraconazole, ketoconazole, micafungin, miconazole, naftifine, natamycin, nystatin, oxyconazole, ravuconazole, posaconazole, rimocidin, sertaconazole, sulconazole, terbinafine, terconazole, tioconazole, and voriconazole; (9) antiinflammatories, e.g., non-steroidal anti-inflammatory agents, such as aceclofenac, acemetacin, amoxiprin, aspirin, azapropazone, benorilate, bromfenac, carprofen, celecoxib, choline magnesium salicylate, diclofenac, diflunisal, etodolac, etoricoxib, faislamine, fenbufen, fenoprofen, flurbiprofen, ibuprofen, indometacin, ketoprofen, ketorolac, lornoxicam, loxoprofen, lumiracoxib, meclofenamic acid, mefenamic acid, meloxicam, metamizole, methyl salicylate, magnesium salicylate, nabumetone, naproxen, nimesulide, oxyphenbutazone, parecoxib, phenylbutazone, piroxicam, salicyl salicylate, sulindac, sulfinpyrazone, suprofen, tenoxicam, tiaprofenic acid, and tolmetin; (10) antimetabolites, such as folate antagonists, purine analogues, and pyrimidine analogues; (11) anti-platelet agents, such as GPIIb/IIIa blockers (e.g., abciximab, eptifibatide, and tirofiban), P2Y(AC) antagonists (e.g., clopidogrel, ticlopidine and CS-747), cilostazol, dipyridamole, and aspirin; (12) antiproliferatives, such as methotrexate, FK506 (tacrolimus), and mycophenolate mofetil; (13) anti-TNF antibodies or soluble TNF receptor, such as etanercept, rapamycin, and leflunimide; (14) aP2 inhibitors; (15) beta-adrenergic agents, such as carvedilol and metoprolol; (16) bile acid sequestrants, such as questran; (17) calcium channel blockers, such as amlodipine besylate; (18) chemotherapeutic agents; (19) cyclooxygenase-2 (COX-2) inhibitors, such as celecoxib and rofecoxib; (20) cyclosporins; (21) cytotoxic drugs, such as azathioprine and cyclophosphamide; (22) diuretics, such as chlorothiazide, hydrochlorothiazide, flumethiazide, hydroflumethiazide, bendroflumethiazide, methylchlorothiazide, trichloromethiazide, polythiazide, benzothiazide, ethacrynic acid, ticrynafen, chlorthalidone, furosenide, muzolimine, bumetanide, triamterene, amiloride, and spironolactone; (23) endothelin converting enzyme (ECE) inhibitors, such as phosphoramidon; (24) enzymes, such as L-asparaginase; (25) Factor VIIa Inhibitors and Factor Xa Inhibitors; (26) famesyl-protein transferase inhibitors; (27) fibrates; (28) growth factor inhibitors, such as modulators of PDGF activity; (29) growth hormone secretagogues; (30) HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, atavastatin, or visastatin); neutral endopeptidase (NEP) inhibitors; (31) hormonal agents, such as glucocorticoids (e.g., cortisone), estrogens/antiestrogens, androgens/antiandrogens, progestins, and luteinizing hormone-releasing hormone antagonists, and octreotide acetate; (32) immunosuppressants; (33) mineralocorticoid receptor antagonists, such as spironolactone and eplerenone; (34) microtubule-disruptor agents, such as ecteinascidins; (35) microtubule-stabilizing agents, such as pacitaxel, docetaxel, and epothilones A-F; (36) MTP Inhibitors; (37) niacin; (38) phosphodiesterase inhibitors, such as PDE III inhibitors (e.g., cilostazol) and PDE V inhibitors (e.g., sildenafil, tadalafil, and vardenafil); (39) plant-derived products, such as vinca alkaloids, epipodophyllotoxins, and taxanes; (40) platelet activating factor (PAF) antagonists; (41) platinum coordination complexes, such as cisplatin, satraplatin, and carboplatin; (42) potassium channel openers; (43) prenyl-protein transferase inhibitors; (44) protein tyrosine kinase inhibitors; (45) renin inhibitors; (46) squalene synthetase inhibitors; (47) steroids, such as aldosterone, beclometasone, betamethasone, deoxycorticosterone acetate, fludrocortisone, hydrocortisone (cortisol), prednisolone, prednisone, methylprednisolone, dexamethasone, and triamcinolone; (48) TNF-alpha inhibitors, such as tenidap; (49) thrombin inhibitors, such as hirudin; (50) thrombolytic agents, such as anistreplase, reteplase, tenecteplase, tissue plasminogen activator (tPA), recombinant tPA, streptokinase, urokinase, prourokinase, and anisoylated plasminogen streptokinase activator complex (APSAC); (51) thromboxane receptor antagonists, such as ifetroban; (52) topoisomerase inhibitors; (53) vasopeptidase inhibitors (dual NEP-ACE inhibitors), such as omapatrilat and gemopatrilat; and (54) other miscellaneous agents, such as, hydroxyurea, procarbazine, mitotane, hexamethylmelamine, and gold compounds.

The compounds provided herein can also be provided as an article of manufacture using packaging materials well known to those of skill in the art. See, e.g., U.S. Pat. Nos. 5,323,907; 5,052,558; and 5,033,252. Examples of pharmaceutical packaging materials include, but are not limited to, blister packs, bottles, tubes, inhalers, pumps, bags, vials, containers, syringes, and any packaging material suitable for a selected formulation and intended mode of administration and treatment.

Provided herein also are kits which, when used by the medical practitioner, can simplify the administration of appropriate amounts of active ingredients to a subject. In certain embodiments, the kit provided herein includes a container and a dosage form of a compound provided herein, including a single enantiomer, a racemic mixture, a mixture of diastereomers, or an isotopic variant thereof; or a pharmaceutically acceptable salt, solvate, or prodrug thereof.

In certain embodiments, the kit includes a container comprising a dosage form of the compound provided herein, including a single enantiomer, a racemic mixture, a mixture of diastereomers, or an isotopic variant thereof; or a pharmaceutically acceptable salt, solvate, or prodrug thereof, in a container comprising one or more other therapeutic agent(s) described herein.

Kits provided herein can further include devices that are used to administer the active ingredients. Examples of such devices include, but are not limited to, syringes, needle-less injectors drip bags, patches, and inhalers. The kits provided herein can also include condoms for administration of the active ingredients.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

The disclosure will be further understood by the following non-limiting examples.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification: kg (kilograms); g (grams); mg (milligrams); μg (micrograms); ng (nanograms); L (liter); mL (milliliters); μL (microliters); mM (millimolar); μM (micromolar); nM (nanomolar); pM (picomolar); h, hr, or hrs (hours); and QD (once a day).

### Example 1

### Suspension Formulations of the Compound for Oral Administration

Two concentrations (1 mg/mL and 10 mg/mL) of expresso flavored suspension formulations of the Compound were prepared.

An oral suspension formulation vehicle comprising 20% (v/v) expresso flavoring syrup and 0.5% (w/v) methylcellulose (METHOCEL® A4M premium, the Dow Chemical Company) in water was first prepared by (i) adding methylcellulose (1.0 g) to purified water (60 mL) at about 90° C. while stirring to form a well dispersed methylcellulose mixture; (ii) adding expresso syrup (40 mL) to purified water (100 mL) to form a expresso syrup solution; and (iii) adding the expresso syrup solution to the methylcellulose mixture in an ice bath with stirring to form the oral suspension formulation vehicle.

Spray dried dispersion of the Compound comprising 25% by weight of the Compound and 75% by weight of polyvinyl pyrrolidone (PVP-K30) was obtained by spraying a solution containing 5% by weight of the Compound, 15% by weight of PVP-K30, 65% by weight of THF, and 16% by weight of methanol.

An oral suspension formulation of the Compound (10 mg/mL) was prepared by (i) transferring 520 mg of the spray dried dispersion of the Compound (130 mg) to a mortar; (ii) adding approximately 0.5 mL of the oral suspension formulation vehicle (13 mL) to the mortar dropwise and using a pestle to wet the solids; (iii) repeating step (ii) until a well dispersed suspension was obtained; and (iv) adding the remaining oral suspension formulation vehicle in 2 mL aliquots while stirring/grinding with a pestle to form the oral suspension formulation of the Compound.

An oral suspension formulation of the Compound (1 mg/mL) that comprises the spray dried dispersion of the Compound and CAR-O-SIL® M5P (4 mg/mL) in 0.5% methylcellulose and 20% expresso flavored syrup was also prepared using the same procedure.

### Example 2

### Dose Selections for Human Clinical Trials

Twenty-eight day toxicity studies in mice and cynomolgus monkeys at doses of the Compound up to 250 mg/kg/day did not define any toxicological target in either animal species. Nearly all parameters were within normal limits at all doses in both animal species.

The sole exceptions were limited to a statistically significant increase in serum alanine aminotransferase (ALT) in female mice dosed at 250 mg/kg/day and a minimal to mild diffuse hypertrophy of centrilobular hepatocytes in the liver of males and females dosed at 250 mg/kg/day. Mean ALT values at Day 29 were 88.4 U/L in the group treated with the Compound as compared to 38.0 U/L in the control group, and were reversible upon study drug discontinuation. Neither of the above observations in mice was considered to be adverse.

Safety margins were calculated based upon proposed human doses in the first-in-human trial and human equivalent dose (HED) calculations derived from the two 28-day toxicology studies in mice and monkeys. This approach is consistent with the United States Food and Drug Administration Guidance for Industry entitled “Estimating the Maximum Safe Starting Dose in Initial Clinical Trials for Therapeutics in Adult Healthy Volunteers,” July 2005.

A no observable adverse effect level (NOAEL) of 250 mg/kg/day of the Compound was reported in the 28-day mouse study and no observable effect levels (NOELs) of 150 and 250 mg/kg/day of the Compound were reported in the 28-day mouse and monkey studies, respectively. However, increases in systemic exposure with dose were limited in both studies and there was little or no increase in plasma levels of the Compound above a dose of 150 mg/kg/day in the 28-day mouse study, or 50 mg/mg/kg in the 28-day monkey study. Therefore, it was deemed appropriate to employ the 150 mg/kg/day dose of the Compound in the mouse and the 50 mg/kg/day dose of the Compound in the monkey to calculate the respective HED values and establish conservative safety margins for the first-in-human trial. Nonetheless, at the proposed first dose of 5 mg of the Compound in healthy human subjects and 1 mg in HCV-infected patients, the respective safety margin estimates were 153- and 610-fold in mice, and 204- and 807-fold in monkeys as shown in Table 1. At the proposed top dose of 100 mg in human subjects, HED-derived safety margins remained about 10-fold in the monkey and about 7-fold in the mouse.

TABLE 1
Determination of Safety Margins of the Compound

| | |
|---|---|
| Proposed Human Dose | Margin of Safety |

| (mg/subject/day) | (mg/kg/day)^{a} | Mouse^{b} | Monkey^{c} |
|---|---|---|---|
| 1 | 0.02 | 610 | 807 |
| 5 | 0.08 | 153 | 204 |
| 10 | 0.17 | 72 | 95 |
| 25 | 0.42 | 29 | 38 |
| 50 | 0.83 | 15 | 19 |
| 100 | 1.67 | 7 | 10 |

^{a}The mean human body weight is assumed to be 60 kg.
^{b}HED was calculated to be 12.20 mg/kg/day by dividing 150 mg/kg/day in the 28-day toxicology study in mice by a correction factor of 12.3.
^{c}HED was calculated to be 16.13 mg/kg/day by dividing 50 mg/kg/day in the 28-day toxicology study in monkeys by a correction factor of 3.1.

### Example 3

### Phase I/IIa Clinical Trial to Assess Single and Multiple Doses of the Compound in Healthy and HCV-Infected Subjects

The phase I/IIa study was designed to evaluate safety and tolerability, pharmacokinetics (PK), food effect, and antiviral activity with regard to the Compound. The key safety parameters that were evaluated in the trial include adverse events (AEs), physical examination, vital signs, 12-lead electrocardiogram (ECG), and standard safety laboratory tests. The key antiviral activity parameters that were evaluated in the trial include changes in plasma HCV RNA and emergence of resistance mutations. The key pharmacokinetic parameters that were evaluated in the trial include plasma and urine concentrations of the Compound.

In the phase I/IIa study, plasma HCV RNA was quantitated using a validated commercial assay (COBAS® TAQMAN®) with a lower limit of quantitation of 25 IU/mL. HCV RNA genotyping was performed at Baseline using the Versant HCV Genotype Assay (LiPA) 2.0. All but one subject were confirmed by direct sequencing. Plasma concentrations of the Compound were quantitated using validated liquid chromatographic methods with mass-spectrometric detection (LC/MS/MS) with a lower limit of quantitation of 0.1 ng/mL.

### Group A

The Compound was first evaluated in a randomized, double-blind, placebo-controlled, sequential dose escalation study in 48 healthy subjects. The dose escalation schedule is summarized in Table 2. For each cohort, there were 6 subjects to receive the Compound and 2 subjects to receive placebo. Except cohort 5a, the Compound was administered under fasted conditions.

TABLE 2
Study Design for Group A

| Cohort | Dose (mg) | N^{#} | Drug Administration |
|---|---|---|---|
| 1a | 5 | 6:2 | The Compound or placebo x 1 day |
| 2a | 10 | 6:2 | The Compound or placebo x 1 day |
| 3a | 25 | 6:2 | The Compound or placebo x 1 day |
| 4a | 50 | 6:2 | The Compound or placebo x 1 day |
| 5a | 50 (food effect) | 6:2 | The Compound or placebo on days 1 |
| | | | and 8 |
| 6a | 100 | 6:2 | The Compound or placebo x 1 day |
| 7a | 100 | 6:2 | The Compound or placebo x 7 days |

^{#}Active:placebo

Dose escalations in Groups A occurred sequentially after safety and PK review between ascending dose cohorts. Given that no maximum tolerated dose was attained and no toxicology targets were defined in the two 28-day toxicology studies, doses would not be escalated once the mean exposures of the Compound approached those which were achieved in the 28-day GLP toxicology study in monkeys. If mean Cₘₐₓ>650 ng/mL or mean AUCₗₐₛₜ>7600 ng·hr/mL were achieved in human subjects, no dose escalation occurred.

For the food effect study (cohort 5a), subjects received a single dose of 50 mg of the Compound or placebo on Days 1 and 8, with a 7 day washout between dosing periods. In the food effect study, the subjects were randomized among two groups, differing by fasted/fed dosing sequences as shown in Table 3. Within each group, subject were also randomized to treatment assignment at a ratio of 3:1 (active:placebo).

TABLE 3
Food Effect Study

| N | Day 1 Dosing | Day 8 Dosing |
|---|---|---|
| 4 | Fasted treatment | Fed treatment |
| 4 | Fed treatment | Fasted treatment |

Forty-eight healthy subjects were studied in Group A and their demographic and baseline characteristics are summarized in Table 4.

### Group B

In Group B, the Compound was evaluated in an open-label, single day study in 18 subjects with chronic HCV Genotype 1, 2, or 3 infection. The dose schedule is summarized in Table 5. The subjects in Group B had compensated liver disease and naive to antiviral treatment, having HCV RNA of no less than 5 log₁₀ IU/mL, ALT of no greater than 5× upper limit of normal, and no other clinically significant laboratory abnormalities. For all cohorts, dosing was under fasted in this study.

TABLE 4

| | | | 5 mg x | | 10 mg x | | 25 mg x | | 50 mg x | | 100 mg x | | 100 mg QD x | |
|---|---|---|---|---|---|---|---|---|---|---|---|---|---|---|
| | Placebo¹ | | 1 day | | 1 day | | 1 day | | 2 days² | | 1 day | | 7 days | |
| | N = 12 | | N = 6 | | N = 6 | | N = 6 | | N = 6 | | N = 6 | | N = 6 | |
| Age, years | | | | | | | | | | | | | | |
| Mean (SE) | 36.4 | (2.78) | 40.8 | (4.93) | 37.3 | (5.38) | 32.7 | (3.37) | 39.3 | (4.86) | 36.7 | (4.62) | 34.0 | (4.23) |
| Sex, n (%) | | | | | | | | | | | | | | |
| Male | 9 | (75.0) | 3 | (50.0) | 2 | (33.3) | 2 | (33.3) | 3 | (50.0) | 2 | (33.3) | 3 | (50.0) |
| Female | 3 | (25.0) | 3 | (50.0) | 4 | (66.7) | 4 | (66.7) | 3 | (50.0) | 4 | (66.7) | 3 | (50.0) |
| Race, n (%) | | | | | | | | | | | | | | |
| White | 5 | (41.7) | 3 | (50.0) | 4 | (66.7) | 1 | (16.7) | 5 | (83.3) | 3 | (50.0) | 3 | (50.0) |
| Black | 5 | (41.7) | 3 | (50.0) | 1 | (16.7) | 5 | (83.3) | 1 | (16.7) | 3 | (50.0) | 3 | (50.0) |
| Asian | 2 | (16.7) | 0 | | 0 | | 0 | | 0 | | 0 | | 0 | |
| Native American | 0 | | 0 | | 1 | (16.7) | 0 | | 0 | | 0 | | 0 | |
| BMI, kg/m² | | | | | | | | | | | | | | |
| Mean (SE) | 26.47 | (0.932) | 25.75 | (0.866) | 27.42 | (1.368) | 25.33 | (1.352) | 26.93 | (0.940) | 28.17 | (1.551) | 24.77 | (1.412) |

¹Data for placebo subjects were pooled for all Group A cohorts.
²Food effect study, dosed on Days 1 and 8.

TABLE 5
Study Design for Group B

| Cohort | Dose (mg) | Genotype | N | Drug Administration |
|---|---|---|---|---|
| 1b | 1 | 1 | 2 | The Compound x 1 day |
| | 5 | | 2 | The Compound x 1 day |
| 2b | 10 | 1 | 2 | The Compound x 1 day |
| 3b | 25 | 1 | 2 | The Compound x 1 day |
| | | 2 or 3 | 2 | The Compound x 1 day |
| 4b | 50 | 1 | 2 | The Compound x 1 day |
| | | 2 or 3 | 2 | The Compound x 1 day |
| 5b | 100 | 1 | 2 | The Compound x 1 day |
| | | 2 or 3 | 2 | The Compound x 1 day |

Eighteen subjects were studied in Group B and their demographic and baseline characteristics are summarized in Table 6.

In Groups A and B, there were no treatment-emergent serious adverse events (SAEs), treatment discontinuations due to an AE or laboratory toxicity. Additionally, no subject met protocol-defined criteria for a dose-limiting toxicity. Furthermore, there were no dose-related, or other patterns of AEs, ECGs, vital signs or newly occurring or worsening graded hematology, chemistry, or urinalysis abnormalities. All AEs were mild to moderate in intensity.

As summarized in Table 7, single dose of the Compound demonstrated potent pan-genotypic antiviral activity in Genotype 1, 2, and 3 HCV-infected subjects. Maximum viral load reductions were typically achieved 24 to 72 hours post-dose.

TABLE 6

| | 1 mg x | | 5 mg x | | 10 mg x | | 25 mg x | | 50 mg x | | 100 mg x | |
|---|---|---|---|---|---|---|---|---|---|---|---|---|
| | 1 day | | 1 day | | 1 day | | 1 day | | 1 day | | 1 day | |
| | N = 2 | | N = 2 | | N = 2 | | N = 4 | | N = 4 | | N = 4 | |
| Age, years | | | | | | | | | | | | |
| Mean (SE) | 56.0 | (2.0) | 44.5 | (6.5) | 50.5 | (0.5) | 55.0 | (3.2) | 50.8 | (1.7) | 49.5 | (4.2) |
| Sex, n (%) | | | | | | | | | | | | |
| Male | 2 | (100) | 2 | (100) | 1 | (50) | 3 | (75) | 4 | (100) | 3 | (75) |
| Female | 0 | | 0 | | 1 | (50) | 1 | (25) | 0 | | 1 | (25) |
| Race, n (%) | | | | | | | | | | | | |
| White | 2 | (100) | 2 | (100) | 1 | (50) | 4 | (100) | 4 | (100) | 3 | (75) |
| Black | 0 | | 0 | | 1 | (50) | 0 | | 0 | | 1 | (25) |
| BMI, kg/m² | | | | | | | | | | | | |
| Mean (SE) | 26.0 | (1.4) | 25.7 | (1.5) | 31.7 | (2.0) | 27.1 | (2.2) | 28.5 | (1.7) | 25.5 | (1.8) |
| HCV RNA, log₁₀ IU/mL | | | | | | | | | | | | |
| Mean (SE) | 6.46 | (0.57) | 7.24 | (0.18) | 5.56 | (0.21) | 6.77 | (0.20) | 6.95 | (0.23) | 6.82 | (0.29) |
| HCV genotype | | | | | | | | | | | | |

| | | | | | | |
|---|---|---|---|---|---|---|
| 1a/1b | 1/1 | 2/0 | 1/1 | 2/0 | 1/1 | 1/1 |
| 2¹/2b | 0 | 0 | 0 | 1/0 | 0/1 | 0/1 |
| 3a | 0 | 0 | 0 | 1 | 1 | 1 |
| IL28B genotype, n (%) | | | | | | |
| CC | 2 | 1 | 0 | 2 | 2 | 3 |
| CT | 0 | 1 | 1 | 1 | 1 | 0 |
| TT | 0 | 0 | 1 | 1 | 1 | 1 |

¹Virus could not be subtyped.

TABLE 7
Maximum Viral Load Reduction after Single Dose of the Compound
(log₁₀ IU/mL, per protocol efficacy population)

Genotype

| Single dose | 1a | 1b | 2² | 3² |
|---|---|---|---|---|
| 1 mg | 0.8² | 3.1² | — | — |
| 5 mg | 2.6¹ | — | — | — |
| 10 mg | 2.9² | 3.7² | — | — |
| 25 mg | 3.7¹ | — | 0.4 | 2.2 |
| 50 mg | 2.5² | 3.2² | 3.2 | 3.7 |
| 100 mg | 3.3² | 3.7² | 3.5 | 3.3 |

¹Mean maximum viral load reduction: 2 subjects/cohort
²Maximum viral load reduction: 1 subject/cohort

TABLE 8

| Dose | PK | Cₘₐₓ | Tₘₐₓ | AUC_{0-inf} | t_{1/2} | C_{trough} | |
|---|---|---|---|---|---|---|---|
| (mg) | Day | (ng/mL) | (h) | (ng · h/mL) | (h) | (ng/mL) | |
| 5 | 1 | 5.1 ± 1.1 | 4.0 (4.0-6.0) | 110 ± 35.4 | 21 ± 4.5 | 1.6 ± 0.5 | (1.0-2.2) |
| 10 | 1 | 11.5 ± 4.3 | 5.0 (4.0-6.0) | 259 ± 123.1 | 24 ± 4.7 | 3.7 ± 1.8 | (1.8-6.3) |
| 25 | 1 | 26.5 ± 11.1 | 4.0 (3.0-6.0) | 570 ± 200.0 | 24 ± 3.2 | 8.1 ± 2.9 | (5.3-11.4) |
| 50 | 1 | 50.9 ± 18.1 | 4.0 (3.0-6.0) | 952 ± 335.8 | 22 ± 1.7 | 13.4 ± 4.6 | (6.0-18.2) |
| 50 (fed) | 1 | 13.2 ± 3.3 | 6.0 (4.0-8.0) | 335 ± 53.9 | 23 ± 3.0 | 5.6 ± 1.3 | (4.0-7.2) |
| 100 | 1 | 83.3 ± 49.1 | 4.0 (3.0-6.0) | 1290 ± 401.6 | 23 ± 2.7 | 17.1 ± 6.2 | (12.0-28.5) |
| 100 QD x | 1 | 86.9 ± 25.9 | 4.0 (3.0-6.0) | 980 ± 312.4¹ | NA | 20.4 ± 9.0 | (9.0-34.7) |
| 7 days | 7 | 117.2 ± 39.4 | 3.5 (3.0-4.0) | 1482 ± 489.2¹ | 21 ± 3.4 | 34.0 ± 14.4 | (16.4-51.4) |

Values are reported as mean ± SD, except for Tₘₐₓ where medians (min-max) are reported.
For C_{trough}, (min-max) is also shown.
NA = not applicable.
¹For 100 mg QD Day 1, AUC₀₋₂₄ is shown.

The pharmacokinetics of the Compound after single and multiple doses in healthy volunteers is summarized in Table 8. Dose proportional exposure was observed in the dose range studied (5 to 100 mg). Following QD dosing×7 days, AUC_{0-inf} was increased by approximately 50% upon reaching steady state; and C_{trough} was increased by about 70% to 34 ng/mL. At 50 mg, exposure was reduced by approximately two-thirds after high-fat (about 55 g), high-calorie (about 950 kcal) meal.

The pharmacokinetics of the Compound after single, fasted, dose in Genotypes 1, 2, and 3 HCV-infected subjects is summarized in Table 9. Dose proportional exposure was observed in the dose range studied (5 to 100 mg). The PK results support QD dosing. Following QD dosing×7 days, AUC_{0-inf} was increased by approximately 50% upon reaching steady state; and C_{trough} was increased by about 70% to 34 ng/mL. At 50 mg, exposure was reduced by approximately two-thirds after high-fat (about 55 g), high-calorie (about 950 kcal) meal.

TABLE 9

| Dose | PK | Cₘₐₓ | Tₘₐₓ | AUC_{0-inf} | t_{1/2} | C_{trough} | |
|---|---|---|---|---|---|---|---|
| (mg) | Day | (ng/mL) | (h) | (ng · h/mL) | (h) | (ng/mL) | |
| 1 | 1 | 0.5 ± 0.4 | 4.0 (4.0-4.0) | 8 ± 5.8 | 13 ± 6.0 | 0.2¹ | (0.15-0.15) |
| 5 | 1 | 2.2 ± 1.1 | 4.0 (4.0-4.0) | 39 ± 14.5 | 13 ± 0.5 | 0.5 ± 0.2 | (0.4-0.6) |
| 10 | 1 | 9.0 ± 1.2 | 3.0 (2.0-4.0) | 188 ± 3.7 | 24 ± 5.9 | 2.6 ± 0.0 | (2.5-2.6) |
| 25 | 1 | 13.8 ± 3.8 | 3.5 (2.0-6.0) | 216 ± 80.6 | 18 ± 3.8 | 2.8 ± 1.1 | (2.0-4.4) |
| 50 | 1 | 35.2 ± 16.5 | 4.0 (4.0-4.0) | 576 ± 299.2 | 22 ± 1.8 | 7.6 ± 4.1 | (4.0-13.1) |
| 100 | 1 | 95.0 ± 20.4 | 4.0 (3.0-4.0) | 1341 ± 281.5 | 19 ± 2.0 | 18.9 ± 5.6 | (12.0-24.2) |

Values are reported as mean ± SD, except for Tₘₐₓ where medians (min-max) are reported.
For C_{trough}, (min-max) is also shown.
¹n = 1

### Group C

In Group C, the Compound was evaluated in a randomized, double-blind, placebo-controlled, parallel dosing study in 34 treatment-naive, genotype 1 HCV-infected subjects. The dose schedule is summarized in Table 10.

TABLE 10
Study Design for Group C

| N^{#} | Genotype | Treatment | | Drug Administration |
|---|---|---|---|---|
| 8:2 | 1 | 25 | mg QD | The Compound (25 mg) QD or |
| | | | | placebo QD x 3 days |
| 8:2 | 1 | 50 | mg QD | The Compound (50 mg) QD or |
| | | | | placebo QD x 3 days |
| 6:1 | 1 | 50 | mg BID | The Compound (50 mg) BID or |
| | | | | placebo BID x 3 days |
| 6:1 | 1 | 100 | mg QD | The Compound (100 mg) QD or |
| | | | | placebo QD x 3 days |

^{#}active:placebo.

Thirty-four HCV-infected Genotype 1 subjects were studied in Group C and their demographic and baseline characteristics (HCV-infected Genotype 1) are summarized in Table 11. Eighty-five percent (29/34) were infected with HCV Genotype 1a, and approximately two thirds (23/34) were IL28B Genotype CT or TT.

### Group D

In Group D, the Compound was evaluated in an randomized, double-blind, placebo-controlled, parallel dosing study in 30 treatment-naive, genotypes 2, 3, and 4 HCV-infected subjects. The dose schedule is summarized in Table 12.

Thirty HCV-infected Genotypes 2, 3, and 4 subjects were studied in Group D and their demographic and baseline characteristics (HCV-infected Genotypes 2, 3, and 4) are summarized in Table 13. Approximately two thirds (21/30) were IL28B Genotype CT or TT.

The subjects in Groups C and D had compensated liver disease and naive to antiviral treatment, HCV RNA of no less than 5 log₁₀ IU/mL, ALT of no greater than 5× upper limit of normal, and no other clinically significant laboratory abnormalities. For all cohorts, dosing was under fasted in this study.

In Groups C and D, there were no safety-related discontinuations or serious adverse events. The most common adverse events included headache, constipation, and nausea. All events were mild or moderate in intensity. There were no apparent patterns or dose dependence of adverse events, laboratory parameters, or EGCs.

TABLE 11

| | Placebo¹ | 25 mg QD | 50 mg QD | 50 mg BID | 100 mg QD |
|---|---|---|---|---|---|
| | x 3 day | x 3 day | x 3 day | x 3 day | x 3 day |
| | N = 6 | N = 8 | N = 8 | N = 6 | N = 6 |
| Age, years | | | | | |
| Mean (SE) | 46.2 (5.53) | 45.1 (4.18) | 48.8 (3.19) | 49.3 (3.48) | 43.5 (3.27) |
| Sex, n (%) | | | | | |
| Male | 3 (50.0) | 5 (62.5) | 7 (87.5) | 5 (83.3) | 5 (83.3) |
| Female | 3 (50.0) | 3 (37.5) | 1 (12.5) | 1 (16.7) | 1 (16.7) |
| Race, n (%) | | | | | |
| White | 6 (100.0) | 7 (87.5) | 7 (87.5) | 4 (66.7) | 4 (66.7) |
| Black | 0 | 1 (12.5) | 1 (12.5) | 2 (33.3) | 2 (33.3) |
| BMI, kg/m2 | | | | | |
| Mean (SE) | 27.3 (1.42) | 26.1 (1.67) | 26.9 (1.09) | 24.6 (1.18) | 27.5 (1.18) |
| HCV RNA, log₁₀ IU/mL | | | | | |
| Mean (SE) | 6.7 (0.22) | 6.6 (0.18) | 5.9 (0.12) | 6.3 (0.13) | 6.4 (0.21) |
| HCV genotype | | | | | |
| 1a | 6 | 5 | 6 | 6 | 6 |
| 1b | 0 | 3 | 2 | 0 | 0 |
| IL28B genotype | | | | | |
| CC | 3 | 3 | 3 | 1 | 1 |
| CT | 3 | 4 | 3 | 5 | 4 |
| TT | 0 | 1 | 2 | 0 | 1 |

¹Data for placebo subjects were pooled for all Genotype 1 cohorts.

TABLE 12
Study Design for Group D

| N^{#} | Genotype | Treatment | | Drug Administration |
|---|---|---|---|---|
| 4:1 | 2 | 50 | mg BID | The Compound (50 mg) BID or |
| | | | | placebo BID x 3 days |
| 4:1 | 2 | 100 | mg QD | The Compound (100 mg) QD or |
| | | | | placebo QD x 3 days |
| 4:1 | 3 | 50 | mg BID | The Compound (50 mg) BID or |
| | | | | placebo BID x 3 days |
| 4:1 | 3 | 100 | mg QD | The Compound (100 mg) QD or |
| | | | | placebo QD x 3 days |
| 4:1 | 4 | 50 | mg BID | The Compound (50 mg) BID or |
| | | | | placebo BID x 3 days |
| 4:1 | 4 | 100 | mg QD | The Compound (100 mg) QD or |
| | | | | placebo QD x 3 days |

^{#}active:placebo.

TABLE 13

| | Placebo¹ | 50 mg BID | 100 mg QD |
|---|---|---|---|
| | x 3 day | x 3 day | x 3 day |
| | N = 6 | N = 12 | N = 12 |
| Age, years | | | |
| Mean (SE) | 43.2 (3.49) | 44.8 (3.22) | 41.2 (2.82) |
| Sex, n (%) | | | |
| Male | 5 (83.3) | 7 (58.3) | 9 (75.0) |
| Female | 1 (16.7) | 5 (41.7) | 3 (25.0) |
| Race, n (%) | | | |
| White | 5 (83.3) | 9 (75.0) | 11 (91.7) |
| Black | 1 (16.7) | 2 (16.7) | 1 (8.3) |
| Native Hawaiian² | 0 | 1 (8.3) | 0 |
| BMI, kg/m² | | | |
| Mean (SE) | 29.3 (1.18) | 26.1 (0.85) | 25.5 (0.93) |
| HCV RNA, log₁₀ IU/mL | | | |
| Mean (SE) | 6.0 (0.22) | 6.3 (0.15) | 6.5 (0.17) |
| HCV genotype | | | |
| 2 | 1 | 1 | 1 |
| 2b | 1 | 3 | 3 |
| 3a | 2 | 4 | 4 |
| 4 | 2 | 4 | 4 |
| IL28B genotype, n | | | |
| CC | 1 | 3 | 5 |
| CT | 5 | 6 | 5 |
| TT | 0 | 3 | 2 |

¹Data for placebo subjects were pooled for all Genotype 2, 3 and 4 cohorts.
²Including other Pacific Islander

As summarized in Table 14, QD and BID dosing of the Compound for three days demonstrated antiviral activity across Genotypes 1, 2, 3, and 4 HCV-infected subjects, with the majority of subjects achieving viral load reductions of 3.2 to 4.3 log₁₀ IU/mL. The Compound had comparable antiviral activity at 50 mg BID and 100 mg QD. No subject experienced a rebound (>1.0 log₁₀ IU/mL increase over nadir) during the 3 days of dosing.

Maximum viral load reductions were typically achieved 24 to 72 hours post dose.

TABLE 14
Mean (Range) Maximal HCV RNA Reduction from Baseline (log₁₀ IU/mL)

| | Genotype | | | | |
|---|---|---|---|---|---|
| Dose | 1a | 1b | 2 | 3 | 4 |
| (x 3 days) | N = 29 | N = 5 | N = 10 | N = 10 | N = 10 |
| Placebo | 0.4 (0.3-0.6) | n = 0¹ | 0.4 (0.3-0.5) | 0.6 (0.5-0.6) | 0.6 (0.4-0.7) |
| | n = 6 | | n = 2 | n = 2 | n = 2 |
| 25 mg QD | 3.3 (2.9-3.7) | 3.0 (2.0-4.2) | NA | NA | NA |
| | n = 5 | n = 3 | | | |
| 50 mg QD | 3.6 (3.3-3.9) | 4.3 (4.1-4.5) | NA | NA | NA |
| | n = 6 | n = 2 | | | |
| 50 mg BID | 3.2 (2.7-3.8) | n = 0¹ | 2.0 (0.5-4.0) | 3.3 (2.5-4.6) | 3.9 (3.4-4.5) |
| | n = 6 | | n = 4 | n = 4 | n = 4 |
| 100 mg QD | 3.5 (2.6-4.3) | n = 0¹ | 2.0 (0.3-4.1) | 3.4 (3.1-3.8) | 3.6 (2.3-4.6) |
| | n = 6 | | n = 4 | n = 4 | n = 4 |

¹Genotype 1b HCV-infected subjects were not randomized to these dosing groups.

The pharmacokinetics of the Compound after QD and BID dosing for three days in Genotypes 1, 2, 3, and 4 HCV-infected subjects is summarized in Tables 15 and 16, respectively. Plasma exposures of the Compound were dose-related in HCV-infected subjects. Trough plasma exposures after repeat dosing of 50 and 100 mg total daily doses exceeded by x-fold the protein-binding adjusted EC₉₀ (˜2.3 ng/mL) associated with the least susceptible HCV genotype. Plasma half-life (˜20 hrs) remained consistent across study populations and doses and supports QD or BID dosing. In Tables 15 and 16, values are reported as mean±SD, except for Tₘₐₓ and C_{trough} for which medians (min-max) are reported.

TABLE 15
Summary of Pharmacokinetics of the Compound after QD Dosing
for 3 Days in Genotypes 1, 2, 3, and 4 HCV-infected Subjects

| Dose | HCV | PK | Cₘₐₓ | Tₘₐₓ | AUC₀₋₂₄ | t_{1/2} | | |
|---|---|---|---|---|---|---|---|---|
| (3 days) | GT¹ | Day | (ng/mL) | (hr) | (ng · hr/mL) | (hr) | C_{trough}² (ng/mL) | |
| 25 mg | 1 | 1 | 14 ± 5.19 | 3.8 (3.0-4.0) | 142 ± 44.5 | NA | 2.9 ± 0.98 | (1.8-4.9) |
| QD | | 3 | 20 ± 6.74 | 3.9 (3.0-6.0) | 235 ± 79.9 | 20.8 ± 4.06 | 5.2 ± 2.02 | (3.0-8.2) |
| 50 mg | 1 | 1 | 36 ± 20.0 | 3.6 (3.0-4.0) | 384 ± 204.0 | NA | 6.8 ± 3.68 | (2.8-13.0) |
| QD | | 3 | 32 ± 8.12 | 3.4 (2.0-4.0) | 387 ± 115.0 | 23.0 ± 3.81 | 8.2 ± 2.75 | (5.2-13.2) |
| 100 mg | 1 | 1 | 38 ± 8.6 | 3.7 (3.0-4.0) | 410 ± 122.0 | NA | 9.2 ± 2.73 | (6.7-14.3) |
| QD | | 3 | 59 ± 18.1 | 3.6 (3.0-4.0) | 673 ± 238.0 | 22.9 ± 3.60 | 16.0 ± 6.59 | (6.0-21.0) |
| 100 mg | 2, 3, 4 | 1 | 58 ± 26.7 | 3.5 (2.0-6.0) | 575 ± 261.1 | NA | 11.5 ± 5.25 | (6.1-23.4) |
| QD | | 3 | 68 ± 37.8 | 3.3 (2.0-4.0) | 751 ± 387.0 | 19.3 ± 2.69 | 15.4 ± 6.65 | (6.2-25.9) |

¹NA = not applicable and GT = genotype.
²C_{trough}: C24 h for single dose and QD.

TABLE 16
Summary of Pharmacokinetics of the Compound after BID Dosing
for 3 Days in Genotypes 1, 2, 3, and 4 HCV-infected Subjects

| Dose | HCV | PK | | Cₘₐₓ | Tₘₐₓ | AUC₀₋₁₂ | t_{1/2} | C_{trough}² | |
|---|---|---|---|---|---|---|---|---|---|
| (3 days) | GT¹ | Day | AM/PM | (ng/mL) | (hr) | (ng hr/mL) | (hr) | (ng/mL) | |
| 50 mg | 1 | 1 | AM | 27 ± 16.1 | 3.7 (3.0-4.0) | 194 ± 105 | NA | 9.5 ± 4.62 | (3.0-16.5) |
| BID | | | PM | 20 ± 9.1 | 5.2 (3.0-8.0) | 190 ± 78 | NA | 13.7 ± 6.21 | (5.3-20.4) |
| | | 3 | AM | 40 ± 14.6 | 3.3 (2.0-4.0) | 349 ± 133 | NA | 18.6 ± 6.98 | (9.9-28.0) |
| | | | PM | 25 ± 9.5 | 3.7 (1.0-8.0) | 260 ± 102 | 19.7 ± 2.0 | 18.5 ± 7.67 | (7.0-27.2) |
| 50 BID | 2, 3, 4 | 1 | AM | 37 ± 18.1 | 3.3 (3.0-4.0) | 259 ± 133 | NA | 12.7 ± 7.82 | (4.5-25.5) |
| | | | PM | 22 ± 6.8 | 6.0 (0-12.0) | 206 ± 67 | NA | 16.4 ± 7.31 | (8.7-29.6) |
| | | 3 | AM | 54 ± 18.7 | 3.0 (2.0-4.0) | 427 ± 164 | NA | 21.9 ± 10.4 | (10.4-42.0) |
| | | | PM | 28 ± 11.2 | 4.1 (1.0-8.0) | 289 ± 122 | 19.7 ±5.7 | 22.0 ± 10.4 | (6.5-43.1) |

¹NA = not applicable; GT = genotype.
²C_{trough}: C12 h for BID.

### Resistance Analysis

Genotype 2 HCV-infected subjects, at a total daily dose of 100 mg for 3 days, had a mean maximal viral load reduction of 2.0 log₁₀ IU/mL. Some genotype 2 subjects had reduced response.

Genotype 2 baseline polymorphisms of the subjects are summarized in Table 17. It was found that pre-existence or emergence of M31 directly correlated with reduced viral load response. As shown in Table 18, L31M substitution in genotype 2 replicon conferred 75-fold resistance.

TABLE 17
Genotype 2 Baseline Polymorphisms

| Variant at Baseline | Total | The Compound | Placebo |
|---|---|---|---|
| M31 | 4 | 3 | 1 |
| L/M31 | 1 | 1 | 0 |
| L31 | 4 | 4 | 0 |

TABLE 18
Effect of Genotype 2 Baseline Polymorphisms on
Viral Load Response to the Compound Treatment

| | | Mean Max Viral Load Response |
|---|---|---|
| Group | Number | (log₁₀ IU/mL) |
| L31 at BL, low VL at Day 4¹ | 2 | −4.06 |
| L31 at BL, L31M at Day 4 | 2 | −2.83 |
| M31 at BL | 3 | −0.45 |

¹Both subjects had a viral load too low to sequence at Day 4 (<1,000 IU/mL)

The Compound was safe and well tolerated at daily doses up to 100 mg for 3 days. The Compound demonstrated potent, pan-genotypic antiviral activity in Genotype 1, 2, 3, and 4 HCV-infected subjects. At a total dose of 100 mg/day for 3 days, mean maximal HCV RNA reductions were approximately 3.5 log₁₀ IU/mL in Genotypes 1, 3, and 4. Genotype 2 HCV-infected subjects at a total daily dose of 100 mg for 3 days had a mean maximal viral load reduction of 2.0 log₁₀ IU/mL. Some Genotype 2 subjects had reduced responses. Pre-existence or emergence of M31 in Genotype 2 HCV-infected subjects directly correlated with reduced viral load response. The NS5A M31 polymorphism is a common Genotype 2 variant that conferred 75-fold resistance in the HCV replicon model. The Compound exhibited dose-related exposure in HCV-infected subjects with trough concentrations supporting once daily dosing of the Compound.

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

1. A method for treating or preventing a hepatitis C virus infection in a human patient, comprising administering to the patient a therapeutically effective amount of [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester or an isotopic variant thereof; or a pharmaceutically acceptable salt or solvate thereof; wherein the therapeutically effective amount is at least 1 mg per day.

2-8. (canceled)

9. The method of claim 1, wherein the therapeutically effective amount is about 5 mg per day, about 10 mg per day, about 25 mg per day, about 50 mg per day, or about 100 mg per day.

10. A method for treating or preventing a hepatitis C virus infection in a human patient, comprising administering to the patient a therapeutically effective amount of [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester or an isotopic variant thereof; or a pharmaceutically acceptable salt or solvate thereof; wherein the therapeutically effective amount is from about 0.02 to about 20 mg/kg/day.

11-16. (canceled)

17. The method of claim 10, wherein the therapeutically effective amount is about 0.02 mg/kg/day, about 0.1 mg/kg/day, about 0.2 mg/kg/day, about 0.5 mg/kg/day, about 1 mg/kg/day, or about 2 mg/kg/day.

18. A method for treating or preventing a hepatitis C virus infection in a human patient, comprising administering to the patient [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester or an isotopic variant thereof; or a pharmaceutically acceptable salt or solvate thereof; in an amount that is sufficient to provide a plasma concentration of the compound at steady state in the range from about 1 nM to about 1 μM.

19-24. (canceled)

25. The method of claim 18, wherein the compound is administered in an amount that is sufficient to provide a plasma concentration of the compound at steady state in the range from about 2 nM to about 100 nM.

26-49. (canceled)

50. The method of claim 1, wherein the virus is a genotype 1 hepatitis C virus.

51. The method of claim 50, wherein the virus is a genotype 1a hepatitis C virus or a genotype 1b hepatitis C virus.

52. (canceled)

53. The method of claim 1, wherein the virus is a genotype 2 hepatitis C virus, a genotype 3 hepatitis C virus, or a genotype 4 hepatitis C virus.

54-62. (canceled)

63. The method of claim 1, wherein the virus is a drug-resistant HCV.

64. The method of claim 63, wherein the drug-resistant HCV is resistant to an anti-HCV agent.

65. The method of claim 64, wherein the anti-HCV agent is an NS5A inhibitor.

66. The method of claim 65, wherein the NS5A inhibitor is BMS-790052.

67. The method of claim 63, wherein the drug-resistant HCV is a genotype 2 drug-resistant hepatitis C virus

68. The method of claim 63, wherein the drug-resistant HCV contains an NS5A protein variant.

69-74. (canceled)

75. The method of claim 1, further comprising administering to the human patient a therapeutically effective amount of a second antiviral agent.

76. The method of claim 75, wherein the second antiviral agent is selected from the group consisting of ribavirin, amantadine, an interleukin, a NS3 protease inhibitor, a cysteine protease inhibitor, a phenathrenequinone, a thiazolidine, a benzanilide, a helicase inhibitor, a polymerase inhibitor, a nucleotide analogue, a liotoxin, acerulenin, an antisense phosphorothioate oligodeoxynucleotide, an inhibitor of IRES-dependent translation, and a ribozyme.

77-78. (canceled)

79. The method of claim 1, wherein the human patient has an IL28B CC genotype, an IL28B CT genotype, or an IL28B TT genotype.

80-82. (canceled)

83. The method of claim 1, wherein the patient treated with the compound has about 1 log₁₀, about 2 log₁₀, about 3 log₁₀, or about 4 log₁₀ reduction in the replication of the virus relative to a patient without the treatment of the compound as determined at 1 day.

84-88. (canceled)

89. A pharmaceutical composition comprising [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester or an isotopic variant thereof; and a solvent, a flavoring agent, an emulsifier, or a thickener.

90-97. (canceled)
